(19) United States
(12) Patent Application Publication
(10) Pub. No.: US 2023/0248379 A1
(43) Pub. Date: Aug. 10, 2023

(54) SYSTEMS AND METHODS TO RESTORE PERFUSION TO A VESSEL

(71) Applicant: Neuravi Limited, Galway (IE)

(72) Inventor: Ray McCARTHY, Galway (IE)

(73) Assignee: Neuravi Limited, Galway (IE)

(21) Appl. No.: 18/108,221

(22) Filed: Feb. 10, 2023

Related U.S. Application Data
(60) Provisional application No. 63/308,829, filed on Feb. 10, 2022.
(60) Provisional application No. 63/443,867, filed on Feb. 7, 2023.

(51) Int. Cl. A61B 17/221; G16H 10/20

(52) U.S. Cl. CPC A61B 17/221; G16H 10/20; A61B 2017/2215

(57) ABSTRACT

Methods and systems for using a clot retrieval device for treating a clot in a blood vessel for use in the treatment of ischemic stroke to achieve a clinically effective revascularization or perfusion rate. Perfusion is restored to the blood vessel by passing the clot retrieval device by, through, or about the thrombus and removing the revascularization device to achieve, on the first pass, approximately a 63.2% final revascularization rate under the modified treatment in cerebral infarction score of equal to or greater than a grade of 2b (mTICI ≥2b) for the plurality of human patients with one or more cerebral occlusions within a predetermined time period of natural stroke symptom onset.

### CROSS-REFERENCE TO RELATED APPLICATIONS

This application claims priority to U.S. Provisional Patent Application No. 63/308,829 filed Feb. 10, 2022. This application also claims priority to U.S. Provisional Patent Application No. 63/443,867 filed Feb. 7, 2023. The contents of these applications are incorporated herein by reference in their entirety as if set forth verbatim.

### FIELD

This disclosure relates to devices and methods of removing acute blockages from blood vessels.

### BACKGROUND

The World Health Organization estimates that 15,000,000 blood clots occur annually. Clots may develop and block vessels locally without being released in the form of an embolus—this mechanism is common in the formation of coronary blockages. Acute obstructions may include blood clots, misplaced devices, migrated devices, large emboli and the like. Thromboembolism occurs when part or all of a thrombus breaks away from the blood vessel wall. This clot is then carried in the direction of blood flow. The large vessels of the brain include the Internal Carotid Artery (ICA), Middle Cerebral Artery (MCA), Vertebral Artery (VA), and the Basilar Artery (BA). Clots can include a range of morphologies and consistencies. Long strands of softer clot material may tend to lodge at bifurcations or trifurcations, resulting in multiple vessels being simultaneously occluded over significant lengths. Older clot material can also be less compressible than softer fresher clots, and under the action of blood pressure it may distend the compliant vessel in which it is lodged. Clots may also vary greatly in length, even in any one given area of the anatomy. For example, clots occluding the middle cerebral artery of an ischemic stroke patient may range from just a few millimeters to several centimeters in length.

Of the 15,000,000 clots that occur annually, one-third of patients die and another one-third are disabled. Two of the primary factors associated with mortality in these patients are the occlusion location and the time to treatment. Large-vessel occlusions present in 46% of unselected acute stroke patients presenting in academic medical centers, are associated with higher stroke severity. These more proximal vessels feed a large volume of brain tissue, ergo clinicians use the presenting NIHSS (National Institute of Health Stroke Scale) score as an indicator of large-vessel occlusion.

With this, it is understood that an ischemic stroke may result if the clot lodges in the cerebral vasculature. It is estimated that 87% of stroke cases are acute ischemic stroke (AIS). In the United States alone, roughly 700,000 AIS cases occur every year and this number is expected to increase with an ageing population. Occlusion of these large arteries in ischemic stroke is associated with significant disability and mortality. Revascularization of intracranial artery occlusions is the therapeutic goal in stroke therapy. Endovascular mechanical revascularization (thrombectomy) is an increasingly used method for intracranial large vessel recanalization in acute stroke. Currently, a number of mechanical recanalization devices are in clinical use. First generation devices included the Merci Retriever device. Newer devices based on stent-like technology, referred to as “stentrievers” or “stent-retrievers”, are currently displacing these first generation thrombectomy devices for recanalization in acute ischemic stroke.

There are significant challenges associated with designing clot removal devices that can deliver high levels of performance. There are also a number of access challenges that make it difficult to deliver devices. For example, the vasculature in the area in which the clot may be lodged is often fragile and delicate and neurovascular vessels are more fragile than similarly sized vessels in other parts of the body and are in a soft tissue bed. Excessive tensile forces applied on these vessels could result in perforations and hemorrhage. Pulmonary vessels are larger than those of the cerebral vasculature, but are also delicate in nature, particularly those more distal vessels.

Stent-like clot retriever devices are being increasingly used to remove clots from cerebral vessels of acute stroke patients, but such devices are not without disadvantages. A stent-like clot retriever relies on its outward radial force to grip the clot. If the radial force is too low, the device will lose its grip on the clot. If the radial force is too high, the device may damage the vessel wall and may require too much force to withdraw. Such devices that have sufficient radial force to deal with all clot types may therefore cause vessel trauma and serious patient injury, and retrievers that have appropriate radial force to remain atraumatic may not be able to effectively handle all clot types. In this respect, retriever devices may differ in size, shape, and physical properties, such as radial force, as discussed above, ease of deployment, friction, radiopacity and interaction with vessel wall. See, Loh Y, Jahan R, McArthur D. Recanalization rates decrease with increasing thrombectomy attempts. American Journal . . . 2010 May;31(5):935-9; and Arai D, Ishii A, Chihara H, Ikeda H, Miyamoto S. Histological examination of vascular damage caused by stent retriever thrombectomy devices, J Neurointery Surg. 2016 Oct;8(10):992-5. Some designs have also been based on in-vitro stroke models that incorporate realistic clot analogs derived from animal blood that represent the wide range of human clots retrieved from stroke patients. See, Eugene F, Gauvrit J-Y, Ferré J-C, Gentric J-C, Besseghir A, Ronzière T, et al. One-year MR angiographic and clinical follow-up after intracranial mechanical thrombectomy using a stent retriever device, AJNR Am J Neuroradiol. 2015 Jan;36(1):126-32 (18), each of which are incorporated by reference herein in their entirety.

Currently, intravenous (IV) lytics are used for patients presenting up to 4.5 hours after symptom onset. Current guidelines recommend administering IV lytics in the 3-4.5 hour window to those patients who meet the ECASS 3 (European Cooperative Acute Stroke Study 3) trial inclusion/exclusion criteria. Since a large percentage of strokes presenting at hospitals are large vessel occlusions, this is an important clinical challenge to address. Additionally, not all patients may be treated with thrombolytic therapy, and so mechanical thrombectomy is a valuable alternative in patients contraindicated to t-PA (tissue plasminogen activator) or where t-PA treatment was not effective.

Further, acute stroke treatment protocols vary by hospital center. Often, computerized tomography (“CT”) is used to exclude hemorrhagic stroke, and CT Angiography is used. Additional imaging assessment, such as MRI or CT Perfusion, varies by center. Recent AIS trials have demonstrated the clinical benefit and reperfusion efficacy of endovascular therapy using stent-retriever devices. See, Zaidat OO, Castonguay AC, Gupta R, Sun CJ, Martin C, Holloway WE, et al. The first pass effect: a new measure for stroke throm-bectomy devices. J Neurolntervent Surg. 2015;7(suppl 1):A2-A3; Chueh JY, Marosfoi MG, Brooks OW, King RM, Puri AS, Gounis MJ. Novel distal emboli protection technology: the EmboTrap. Intery Neurol. 2017;6:268-276. doi: 10.1159/000480668; Kabbasch C, Mpotsaris A, Liebig T, Söderman M, Holtmannspötter M, Cronqvist M, et al. TREVO 2 Trialists. Trevo versus Merci retrievers for thrombectomy revascularisation of large vessel occlusions in acute ischaemic stroke (TREVO 2): a randomised trial. Lancet. 2012;380:1231-1240. doi: 10.1016/S0140-6736(12)61299-9. There are several FDA approved stent retriever devices indicated for neuro-thrombectomy, including Merci®, Trevo®, and Solitaire®. These devices are generally described in U.S. Pat. Nos. 8,066,757; 8,088,140; 8,945,172; 9,320,532; 8,585,713; 8,945,143; 8,197,493; 8,940,003; 9,161,766; 8,679,142; 8,070,791; 8,574,262; 9,387,098; 9,072,537; 9,044,263; 8,795,317; 8,795,345; 8,529,596; and 8,357,179. The results of these trials provide a valid scientific basis for the establishment of a composite performance goal derived using a Bayesian meta-analysis. Presently, these devices are now considered the standard of care for treatment of AIS secondary to large-vessel occlusion. See, Powers WJ, Derdeyn CP, Biller J, Coffey CS, Hoh BL, Jauch EC, et al; American Heart Association Stroke Council. 2015 American Heart Association/American Stroke Association focused update of the 2013 guidelines for the early management of patients with acute ischemic stroke regarding endovascular treatment: a guideline for healthcare professionals from the American Heart Association/American Stroke Association. Stroke. 2015; 46:3020-3035. doi: 10.1161/STR.0000000000000074.

In a pooled, individual participant data meta-analyses of these trials, the rate of successful reperfusion (mTICI ≥2b) was 71%, whereas the rate of final complete reperfusion (mTICI=3) was only 33%. The modified treatment in cerebral ischemia (mTICI) score categorizes the amount of flow restoration after endovascular revascularization. Specifically, the mTICI score was developed from the original Thrombolysis in Cerebral Infarction (TICI) scale by a consensus group in 2013. The recommendations included a name change to better reflect the increasing use of endovascular therapy for stroke, and simplification of the TICI 2 component to less than half of the target vascular territory (mTICI 2a) or more than half (mTICI 2b). Classification: Grade 0: no perfusion; Grade 1: antegrade reperfusion past the initial occlusion, but limited distal branch filling with little or slow distal reperfusion; Grade 2; Grade 2a: antegrade reperfusion of less than half of the occluded target artery previously ischemic territory (e.g. in one major division of the middle cerebral artery (MCA) and its territory); Grade 2b: antegrade reperfusion of more than half of the previously occluded target artery ischemic territory (e.g. in two major divisions of the MCA and their territories); Grade 3: complete antegrade reperfusion of the previously occluded target artery ischemic territory, with absence of visualized occlusion in all distal branches.

It is understood that mention of percentages in this disclosure refer to averages, unless otherwise specified. In other words, there was a 30% rate of failed reperfusion and only one third of patients achieving final complete reperfusion after all interventions. There was also a limited rate of final near complete or complete (mTICI ≥2c) with reported rates up to 50%. The first pass attempts for each device for near complete or complete reperfusion was also still relatively low only being up to 30%. Opportunities also exist to increase the rate of near-complete reperfusion (mTICI ≥2c) from a single pass of a device to improve patient outcomes. For example, for these earlier devices 90-day functional independence rates of 61.3% versus 35.3% in non-FP success have been observed.

In view of these clear performance disadvantages, further reperfusion and patient outcomes advances in AIS treatment are warranted. The solution of this disclosure resolves these and other issues of the art.

### SUMMARY

The subject of this disclosure is the use of a clot retrieval device to treat ischemic stroke for restoring perfusion and/or removing a clot and other obstructions from the neurovascular arteries and veins as well as other vascular beds.

An example of treating an ischemic stroke can include delivering and passing at least one clot retrieval device at least one time through an occluded blood vessel to achieve a clinically effective revascularization rate.

One example can be a method of treating an occluded blood vessel in a human by restoring a clinically effective perfusion rate to the tissue distal of the occluded blood vessel by passing at least one clot retrieval device at least one time through the occluded blood vessel.

In some examples, a method of treating ischemic stroke is disclosed. The method can include removing thrombus in human patients experiencing ischemic stroke; delivering a revascularization device to a blood vessel to retrieve a thrombus of a respective human patient of a plurality of human patients each having one or more cerebral occlusions; and restoring perfusion to the blood vessel by passing the revascularization device by, through, or about the thrombus and removing the revascularization device to achieve, on a first pass of the revascularization device, an outcome of approximately a 63.2% revascularization rate with a cerebral infarction score of equal to or greater than a grade of 2b (mTICI ≥2b) for the plurality of human patients within a predetermined time period of natural stroke symptom onset.

In some examples, a method of treating ischemic stroke is disclosed. The method can include achieving approximately a 38.4% revascularization rate mTICI ≥2c on the first pass of the revascularization device in the blood vessel.

In some examples, a method of treating ischemic stroke is disclosed. The method can include wherein the outcome is for a population size that is at least 985 patients of the plurality of human patients.

In some examples, a method of treating ischemic stroke is disclosed. The method can include the revascularization device having a collapsed delivery configuration and an expanded deployed configuration, the revascularization device comprising a framework of struts forming a porous inner body flow channel and having a tubular main body portion and a distal end; and a framework of struts forming an outer tubular body radially surrounding the tubular main body portion of the inner body during both the collapsed delivery configuration and the expanded deployed configuration, the inner body being connected to the outer tubular body.

In some examples, a method of treating ischemic stroke is disclosed. The method includes the revascularization device further comprising: a shaft extending between a proximal end and a distal end; the outer body being self-expandable and coupled to the shaft, the self-expandable outer body comprising a plurality of longitudinally spaced clot scaffolding segments separated by voids forming one or more clot inlet mouths between adjacent clot scaffolding segments of the plurality of longitudinally spaced clot scaffolding segments; and the inner body flow channel being self-expandable .

In some examples, a method of treating ischemic stroke is disclosed. The method includes wherein the thrombus is located in one of the following locations: a carotid artery, a M1 middle cerebral artery, a M2 middle cerebral artery, a basilar artery, and a vertebral artery.

In some examples, a method of treating ischemic stroke is disclosed. The method includes wherein the plurality of human patients experience a safety outcome of 1.6% based on a 24-hour symptomatic intracerebral hemorrhage (sICH) in cases with the first pass of the revascularization device.

In some examples, a method of treating ischemic stroke is disclosed. The method includes wherein the safety outcome is for a population size of the plurality of human patients that is at least 997 patients.

In some examples, a method of treating ischemic stroke is disclosed. The method includes wherein the plurality of human patients experience a safety outcome of 0.6% based on a 24-hour symptomatic intracerebral hemorrhage (sICH) in cases with a single pass of the revascularization device.

In some examples, a method of treating ischemic stroke is disclosed. The method includes wherein the outcome is for a population size of the plurality of human patients that is at least 504 patients

In some examples, a method of treating ischemic stroke is disclosed. The method includes the plurality of human patients experience a median number of passes of 1 in cases with the first pass of the revascularization device in a population size that is at least 997 patients and a final revascularization rate mTICI ≥2b in the blood vessel after procedure completion.

In some examples, a method of treating ischemic stroke is disclosed. The method includes wherein 85.1% of the plurality of human patients in a population size that is at least 997 patients have less than or equal to 3 passes and a final revascularization rate mTICI ≥2b in the blood vessel after procedure completion.

In some examples, a method of treating ischemic stroke is disclosed. The method includes wherein the outcome comprises a modified Rankin Scale (mRS) of 0 to less than or equal to 2 or less than or equal to baseline at 90 days of approximately 46.8%.

In some examples, a method of treating ischemic stroke is disclosed. The method includes inclusion criteria for the human patients comprising: age ≥18y; informed consent; angiographic confirmation of Large Vessel Occlusion (LVO); clinical decision for mechanical thrombectomy (MT); and the revascularization device planned as first attempted device/technique for MT.

In some examples, a method of treating ischemic stroke is disclosed. The method includes exclusion criteria for the human patients comprising: currently participating in an investigational clinical trial that may confound study endpoints and pregnancy.

In some examples, a method of treating ischemic stroke is disclosed. The method includes delivering a revascularization device to a blood vessel to retrieve a thrombus of a respective human patient of a plurality of human patients each having one or more cerebral occlusions; and restoring perfusion to the blood vessel by passing the clot revascularization device by, through, or about the thrombus and removing the revascularization device to achieve, on a first pass of the revascularization device, a cerebral infarction score of between 2c and 3 (mTICI 2c-3) for the plurality of human patients, with the thrombus composition comprising a von Willebrand Factor percentage of 1.20 to 29.02.

In some examples, a method of treating ischemic stroke is disclosed. The method includes wherein the thrombus composition comprises a von Willebrand Factor percentage of 15.11.

In some examples, a method of treating ischemic stroke is disclosed. The method includes wherein the population size of the plurality of human patients is at least 192 patients.

In some examples, a method of treating ischemic stroke is disclosed. The method includes wherein the population size of the plurality of human patients is at least 314 patients.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 shows a patient catheterized via femoral access with an example clot retrieval device positioned in a cerebral vessel using the arterial system for its delivery.

FIG. 2 shows certain anatomy of cerebral arteries above the aortic arch leading to the brain.

FIG. 3 shows an isometric view of an example stent retriever device of this disclosure.

FIG. 4 is a table summarizing Thrombolysis in Cerebrovascular Infarction (mTICI) inclusive of the 2c rating for the study of this disclosure.

FIG. 5 is a table summarizing Modified Rankin Scale (mRS) score for the study of this disclosure.

FIG. 6 is overview of an example of the device of this disclosure, an EMBOTRAP® II Revascularization Device (“EmboTrap”) as sold by Cerenovus, 33 Technology Dr., Irvine, Calif., USA.

FIG. 7 is a schematic of the study design of this disclosure.

FIG. 8 is an overview of the study based on EmboTrap, where EmboTrap was used in the first line or pass to remove a clot.

FIG. 9 is an overview of the EXCELLENT Registry showing the aims of the study and the methods used.

FIG. 10 is a flow chart showing clot data from 22 US Centers.

FIG. 11 is a table showing the demographics and baseline characteristics from the study and included “All-Comers” where the device was used in the first pass.

FIG. 12 is table showing the continued demographics and baseline characteristics from the study.

FIG. 13 is a table showing the procedural, angiographic, clinical and safety outcomes from the study.

FIG. 14 is a flow chart showing how do clinical outcomes compare for reperfusion with first pass vs later passes.

FIG. 15 is a table showing mTICI 2c-3 for 1 pass and for more than one pass.

FIG. 16 is a chart showing first pass reperfusion and clinical outcomes.

FIG. 17 is a flow chart showing the benefit of first pass reperfusion also observed in the clot population.

FIG. 18 is a table showing mTICI 2c-3 for 1 pass and for more than one pass.

FIG. 19 is a chart showing first pass reperfusion and clinical outcomes.

FIG. 20 is a chart showing first pass reperfusion and clot composition.

FIG. 21 is a table showing mTICI 2c-3 for 1 pass and for more than one pass.

FIG. 22 is a chart showing first pass mTICI 2c-3 data.

FIG. 23 is a chart showing clot composition data.

FIG. 24 is table showing univariate analysis—independent predictors of first pass reperfusion.

FIG. 25 is table showing univariate analysis—independent predictors of first pass reperfusion.

FIG. 26 is table showing multivariate analysis—independent predictors of first pass reperfusion.

FIG. 27 is a summary of the study of this disclosure.

FIG. 28 is a table of demographic and baseline characteristics according to this disclosure.

FIG. 29 is a table summarizing the combined clot composition.

FIG. 30 is a table showing the overall clot composition in the study.

FIG. 31 is an illustration showing clot composition: RBC vs Platelets clot classification and illustrative in vitro clot analogs of analysis cohorts (SEM).

FIG. 32 is a table of the odds ratio of the correlation of certain factors in the blood to the achieved 90 day mRS 0-2 of this disclosure.

FIG. 33 is a table of the 90 day mRS 0-2 of this disclosure.

FIG. 34 is a table of the odds ratio of the correlation of certain factors in the blood to the 90 day all-cause mortality of this disclosure.

FIG. 35 is a table of the 90 day all-cause mortality of this disclosure.

FIG. 36 is a summary of the clinical outcome, safety outcome and procedural outcome of this disclosure.

FIG. 37 is a summary based on median RBC and median platelet content according to this disclosure.

FIG. 38 is a summary of the study design according to this disclosure.

FIG. 39 is a summary of the study endpoints according to this disclosure.

FIG. 40 is a summary of the study inclusion and exclusion criteria of this disclosure.

FIG. 41 is a summary of the study statistical analysis of this disclosure.

FIG. 42 is a flow chart of the Results—Consort Diagram of this disclosure.

FIG. 43 is a table of the demographics and baseline characteristics of this disclosure.

FIG. 44 is a table of the baseline characteristics of this disclosure.

FIG. 45 is a table of the procedural outcomes of this disclosure.

FIG. 46 is a table of the procedural and clinical outcomes of this disclosure.

FIG. 47 is a table of the procedural and clinical outcomes of this disclosure.

FIG. 48 is a summary of the study of this disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, vasculature of a “subject” or “patient” may be vasculature of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include a doctor, surgeon, or any other individual or delivery instrumentation associated with delivery of a clot retrieval device to the vasculature of a subject.

As discussed herein, “thrombus” can be understood as a clot in the circulatory system that remains in a site of the vasculature hindering or otherwise obstructing flow in a blood vessel. The terms, “clot”, “thrombus”, “obstruction”, “occlusion”, “blockage”, and/or the like, can be and are often used interchangeably throughout this disclosure.

Delivery of a “revascularization device” is typically accomplished via delivery of one or more catheters into the femoral artery and/or the radial artery, guided into the arteries of the brain, vascular bypass, angioplasty, and/or the like. “Revascularization devices” can include, but not be limited to, one or more stents, stentrievers, clot removal devices, clot retrieval devices, aspiration systems, one or more combinations thereof, and/or the like, each of which are often used interchangeably throughout this disclosure.

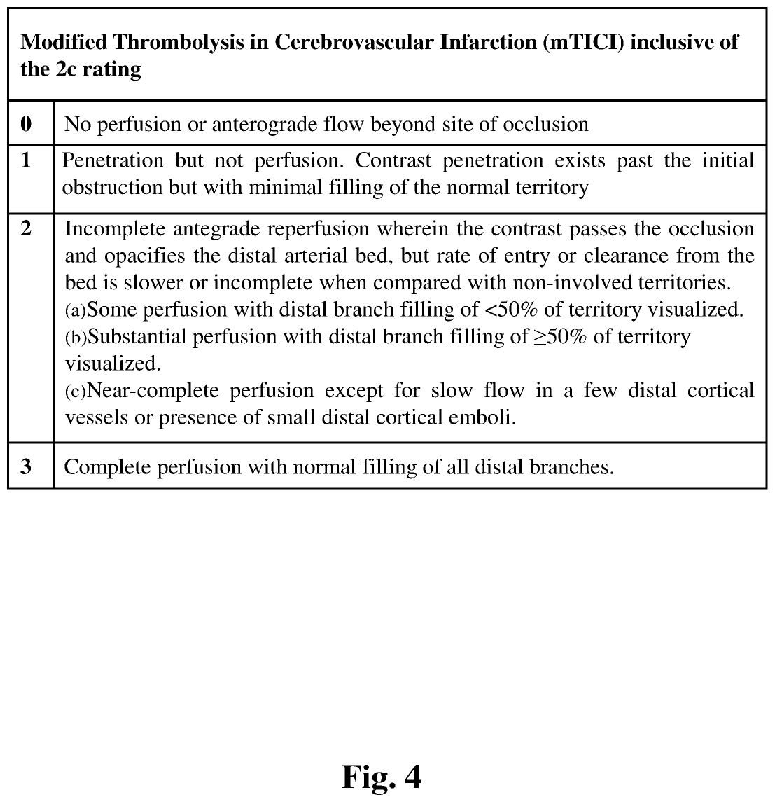

As discussed herein, “mTICI” means modified thrombolysis in cerebral infarction (TICI) score. An mTICI score of 0 means no perfusion. An mTICI score of 1 means antegrade reperfusion past the initial occlusion but limited distal branch filling with little or slow distal reperfusion. An mTICI score of 2 generally means incomplete antegrade reperfusion wherein the contrast passes the occlusion and opacifies the distal arterial bed but there are residual antegrade perfusion deficits. More particularly, an mTICI score of 2a means antegrade reperfusion of less than half of the occluded target artery previously ischemic territory (e.g., in 1 major division of the MCA and its territory). An mTICI score of 2b means antegrade reperfusion of more than half of the previously occluded target artery ischemic territory (e.g., in 2 major divisions of the MCA and their territories). An mTICI score of 2c means antegrade reperfusion of >90%, but less than mTICI 3 or near complete reperfusion. An mTICI score of 3 means full perfusion with filling of all distal branches. This is also illustrated in FIG. 4.

It is noted, however, that other measures of cerebral scoring standards, such as expanded TICI (eTICI), other known and/or to-be-developed cerebral scoring standards, provide measures of cerebral scoring and are thus directly and/or indirectly applicable in understanding scope of the presently disclosed solution. eTICI scale is a 7-point compilation of TICI grades that reflects all previously reported thresholds used to define reperfusion after endovascular stroke therapy. For example, eTICI grade 0, just as mTICI, can be equivalent to no reperfusion or 0% filling of the downstream territory. eTICI 1 can indicate thrombus reduction without any reperfusion of distal arteries, including reperfusion of less than half or 1-49%. eTICI of 2b50 can be 50-66% reperfusion. eTICI 2b67 can be 67-89% reperfusion, exceeding TICI but below TICI2C. eTICI 2c can be equivalent to TICI 2C or 90-99% reperfusion. eTICI 3 can be complete or 100% reperfusion, such as TICI 3. It is understood that one of ordinary skill in the art can also correlate between currently known cerebral scoring standards and/or to-be-developed cerebral scoring standards (e.g., from mTICI to eTICI).

As discussed herein, “NIHSS Score” means The National Institutes of Health Stroke Scale, or NIH Stroke Scale (NIHSS) and is a tool used by healthcare providers to objectively quantify the impairment caused by a stroke. The NIHSS is composed of 11 items, each of which scores a specific ability between a 0 and 4. For each item, a score of 0 typically indicates normal function in that specific ability, while a higher score is indicative of some level of impairment. The individual scores from each item are summed in order to calculate a patient's total NIHSS score. The maximum possible score is 42, with the minimum score being a 0.

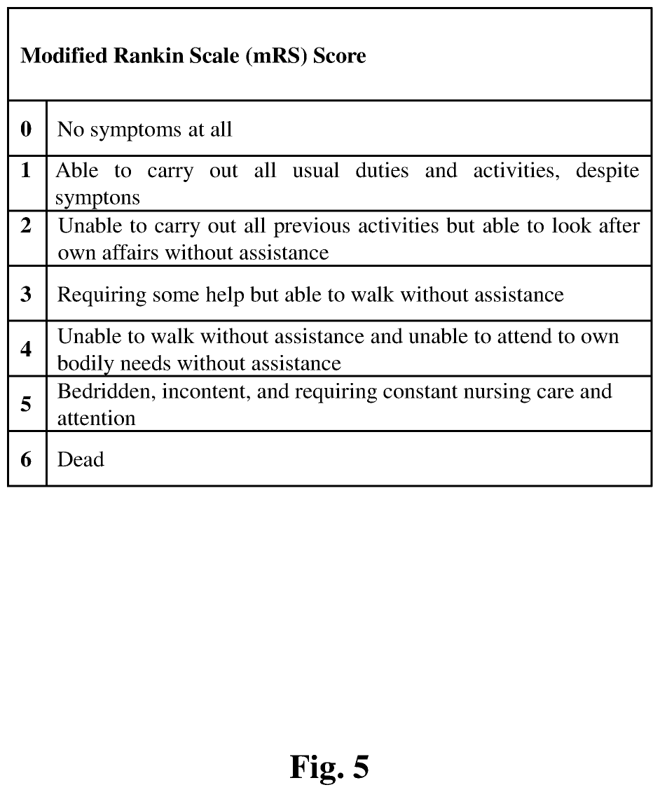

As discussed herein, “mRS” means the modified Rankin Scale (mRS) that is a commonly used scale for measuring the degree of disability or dependence in the daily activities of people who have suffered a stroke or other causes of neurological disability. The mRS scale runs from 0-6, running from perfect health without symptoms to death. An mRS score of 0 is understood as no symptoms being observed. An mRS score of 1 is understood as no significant disability is observed and the patient is able to carry out all usual activities, despite some symptoms. An mRS score of 2 is understood as slight disability and the patient is able to look after own affairs without assistance, but unable to carry out all previous activities. An mRS score of 3 is understood as moderate disability whereby the patient can require some help but is able to walk unassisted. An mRS score of 4 is understood as moderate severe disability and the patient is unable to attend to own bodily needs without assistance or walk unassisted. An mRS score of 5 is understood as severe disability and the patient requires constant nursing care and attention, bedridden, incontinent. An mRS score of 6 is understood as the patient being deceased. This is as illustrated in FIG. 5.

As discussed herein, the term “safety”, as it relates to a clot retrieval device, delivery system, or method of treatment refers to a relatively low severity of adverse events, including adverse bleeding events, infusion or hypersensitivity reactions. Adverse bleeding events can be the primary safety endpoint and include, for example, major bleeding, minor bleeding, and the individual components of the composite endpoint of any bleeding event.

As discussed herein, unless otherwise noted, the term “clinically effective” (used independently or to modify the term “effective”) can mean that it has been proven by a clinical trial wherein the clinical trial has met the approval standards of U.S. Food and Drug Administration, EMEA or a corresponding national regulatory agency. For example, a clinical study may be an adequately sized, randomized, double-blinded controlled study used to clinically prove the effects of the reperfusion device and related systems of this disclosure. Most preferably to clinically prove the effects of the reperfusion device with respect to an ischemic event, for example, to achieve a clinically effective outcome in for the patient suffering the ischemic event (e.g., mRS less than or equal to 2) and/or achieve reperfusion the vessel(s) afflicted by the ischemic event.

As discussed herein, “sICH” is any extravascular blood in the brain or within the cranium associated with clinical deterioration, as defined by an increase of 4 points or more in the score on the NIHSS, or that leads to death and is identified as the predominant cause of the neurologic deterioration. For the purpose of this disclosure, patients with sICH identified through all post-treatment scans up to the 24-hour time-point (including those performed due to clinical deterioration), were considered in the study discussed herein.

As discussed herein, the term “computed tomography” or CT means one or more scans that make use of computer-processed combinations of many X-ray measurements taken from different angles to produce cross-sectional (tomographic) images (virtual “slices”) of specific areas of a scanned object, allowing the user to see inside the object without cutting. Such CT scans of this disclosure can refer to X-ray CT as well as many other types of CT, such as positron emission tomography (PET) and single-photon emission computed tomography (SPECT).

The present disclosure is related to systems, methods and devices restoring perfusion in blood vessels, and in particular clots from cerebral vessels. Certain features, such as a capture net, can be designed to trap a wide range of clot compositions inside the device, and an inner channel to stabilize the clot during retrieval. Certain feature of the retriever of this disclosure can allow the segments to remain open and opposed to the vessel wall while retracted through challenging vessels.

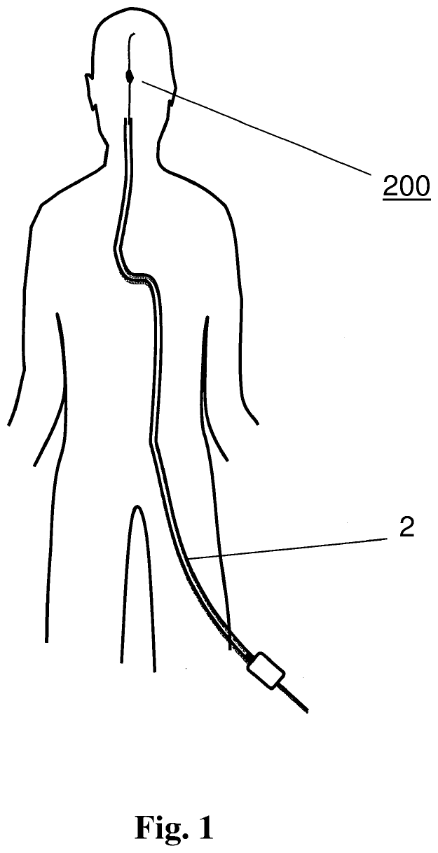

As an example, FIG. 1 depicts a schematic representation of the catheterization of a patient with a clot retrieval device 200, also known as a reperfusion device, via the femoral artery with a catheter 2. Example device 200 is a clinically approved FDA clot retrieval device that can restore blood flow in the neurovasculature by removing thrombus in patients experiencing ischemic stroke within 8 hours of symptom onset. However, it is understood that example device 200 could be used to restore blood flow in less than 8 hours of symptom onset (e.g., 6 hours) or up to 24 hours from symptom onset. As applicable procedure guidelines change with respect to the use of clot retrieval devices for treatment of ischemic events, it is also conceivable that device 200 could be used more than 24 hours from symptom onset. Device 200 can be understood as including features clearly described as incorporated by reference in its entirety from the U.S. Provisional applications from which this application claims priority, which includes U.S. Pat. Nos. 8,777,976; 8,852,205; 9,402,707; 9,445,829; and 9,642,639, each of which are incorporated by reference in their entirety as if set forth verbatim herein. Note that reperfusion devices can also be introduced through the wrist artery (radial access) or directly through the carotid artery. While both radial and carotid access avoids the aortic arches, there are other drawbacks. However, all three approaches are considered to be known to ones of skill in this art.

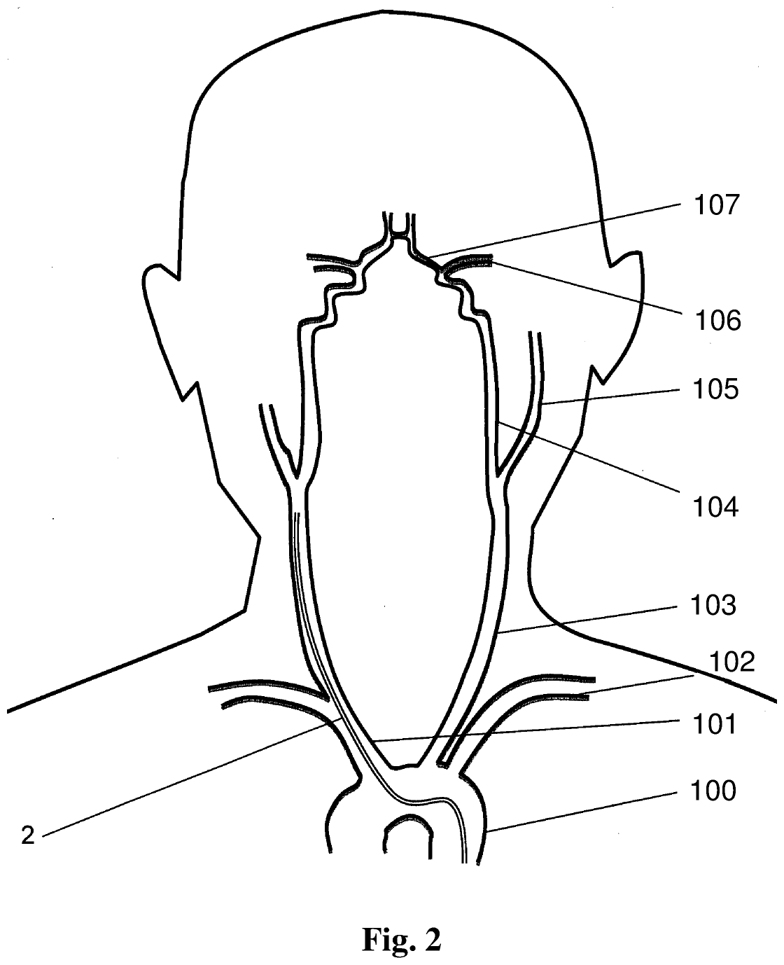

FIG. 2 shows a schematic representation of certain example cerebral vessels. Vessel 100 is the Aorta. Vessel 101 is the brachiocephalic artery. Vessel 102 is the subclavian artery. Vessel 103 is the common carotid artery. Vessel 104 is the internal carotid artery. Vessel 105 is the external carotid artery. Vessel 106 is the middle cerebral artery. Vessel 107 is the anterio-cerebral artery. The catheter 2 from FIG. 1 is shown with its distal end in the common carotid artery. In the more detailed drawings of the invention the details of the access site will not be shown but in general access and delivery is in accordance with FIGS. 1 and 2. Device 200 can be designed for use in the anterior and posterior neurovasculature in vessels such as the internal carotid artery, the M1 and M2 segments of the middle cerebral artery, the vertebral artery, and the basilar arteries. Device 200 can be delivered endovascularly under fluoroscopic guidance in a similar manner to that of other neurovascular clot-retrieval systems.

Once across the site of vessel occlusion, the stent-like element of device 200 is deployed to entrap the clot and allow it to be retrieved, hence restoring blood flow. Device 200 can be a dual-layer stent retriever, with articulating petals, and a distal capture zone for effectively trapping, retaining, and removing various clot types to restore blood flow in patients with AIS secondary to large-vessel occlusion. Examples of the device 200 can be available in two lengths, 5×21 mm and 5×33 mm. It is understood that device 200 of this disclosure would be used with a delivery system to the site of the clot, including a guide catheter, a microcatheter, and/or a guidewire. It is also contemplated that device 200 of this disclosure could be used in connection with an aspiration system to further facilitate restoring perfusion to the vasculature.

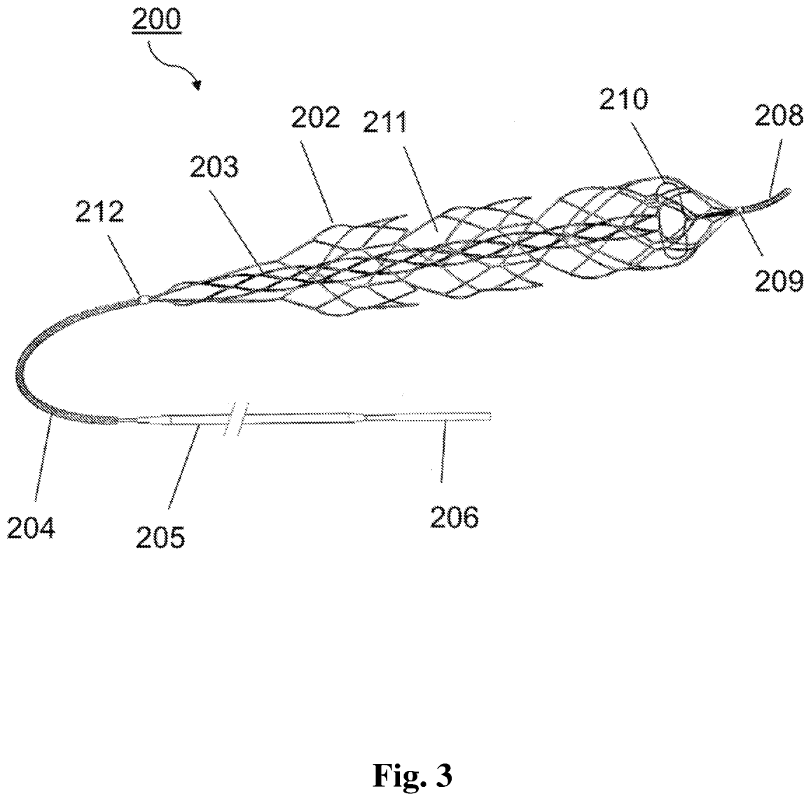

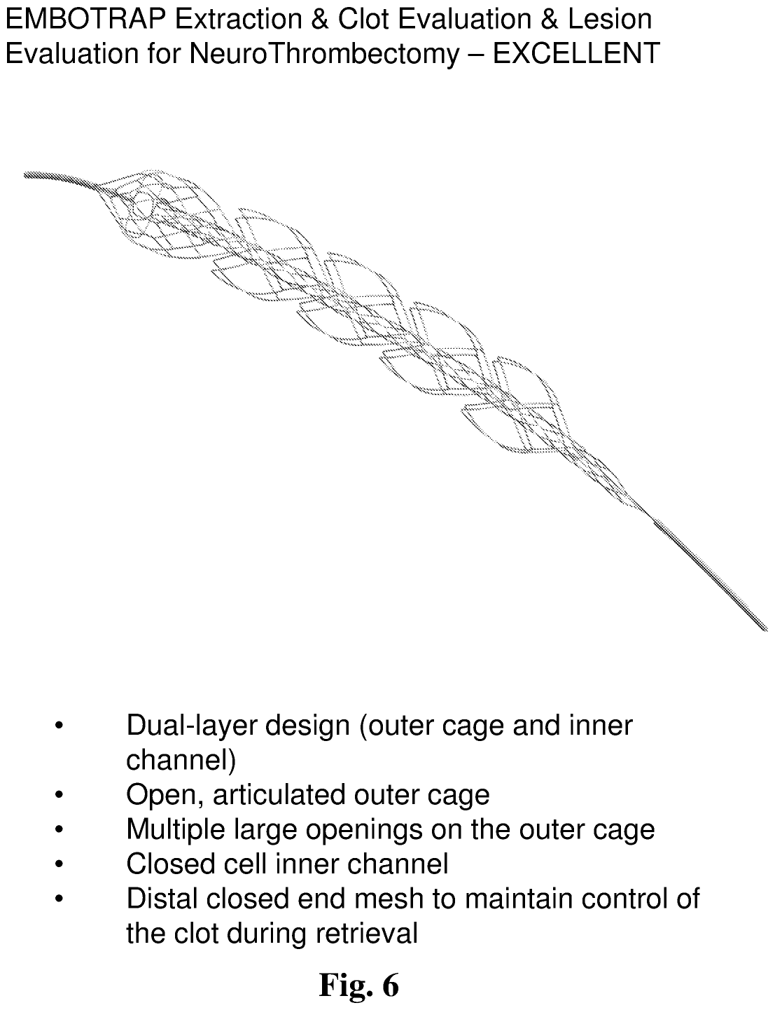

FIGS. 3 and 6 shows one embodiment of an example clot retrieval device of this disclosure. Device 200 can have an elongate shaft 206. Shaft 206 can have a distal end that extends interior of the artery and a proximal end that extends exterior of the artery. Shaft 206 can also have a clot engaging portion configured at its distal end having an outer expandable member 202 and an inner expandable member 203 to facilitate restoration of blood flow through the clot after device 200 is deployed. The inner body flow channel 203 can be self-expandable. A framework of struts forming the porous inner body flow channel 202 can include a tubular main body portion and a distal end. Members 202 and 203 can be configured to have a collapsed configuration for delivery and an expanded configuration for clot retrieval, restoration of perfusion, and fragmentation protection in general. A framework of struts forming an outer tubular body 203 radially surrounds the tubular main body portion of the inner body 202 during both the collapsed delivery configuration and the expanded deployed configuration. The inner body is connected to the outer tubular body.

Shaft 206 may be a tapered wire shaft, and may be made of stainless steel, MP35N, Nitinol or other material of a suitably high modulus and tensile strength. Shaft 206 has a coil 204 adjacent its distal end and proximal of the outer member and inner tubular member. The coil may be coated with a low friction material or have a polymeric jacket positioned on the outer surface. Sleeve 205 may be positioned on shaft 206 adjacent coil 204. Sleeve 205 may be polymeric and may be positioned over the tapered section of shaft 206.

The outer member 202 is tubular and configured to self-expand upon release from a microcatheter to a diameter larger than that of the inner tubular member 203. Expansion of the outer member 202 causes compression and/or displacement of the clot during expansion for purposes of restoring perfusion to the vessel. A radiopaque coil 208 (which may be platinum or gold or an alloy of same) is positioned over the distal end of member 203 and butts against the distal collar 209 of the outer member 202, where it is connected by an adhesive joint to the collar 209. In some examples, the distal end of device 200 at or adjacent collar 209 can be closed by way of struts 210 being joined. In some examples, the outer member 202 can have a closed distal clot capture structure whereby a plurality of struts converge at a terminal connection. In some examples, the distal end of the outer member 202 can have its struts terminate at a distal end in a junction to define a closed end that can prevent egress of clot (or clot fragments that have entered thereof) between the inner 203 and outer 202 members. Inlet openings of outer member 202 can provide the primary movement freedom available to the clot and so the expansion of the outer member 202 urges the clot into the reception space 211 and outer member 202 can have multiple inlet mouths to accept the clot. The self-expandable outer body 202 includes a plurality of longitudinally spaced clot scaffolding segments separated by voids forming one or more clot inlet mouths 211 between adjacent clot scaffolding segments of the plurality of longitudinally spaced clot scaffolding segments. Optionally expanded distal struts 210 can be included with the inner member 203 and function as an additional three-dimensional filter to prevent the egress of clot or clot fragments.

### Study Overview

This disclosure is more clearly understood with a study discussed more particularly below with respect to treatment of ischemic stroke, which is in the Appendix of U.S. Provisional No. 63/443,867, incorporated by reference in its entirety as if set forth verbatim herein from the U.S. Provisional application from which this application claims priority. It is understood that data is presented herein for purposes of illustration and should not be construed as limiting the scope of the disclosed technology in any way or excluding any alternative or additional embodiments.

In each study, device 200 was prepared for delivery to the occlusion site with standard interventional techniques to access the arterial system and using angiography in order to determine the location of the occluded vessel. Device 200 was used pursuant to its instructions for use and used as the first line treatment.

One example for the delivery of device 200 is using a guide catheter, sheath, or balloon guide catheter was advanced as close to the occlusion as possible. A rotating hemostasis valve (RHV) was connected to the proximal end of the catheter and connected to a continuous flush system. An appropriate microcatheter was then selected and an RHV was connected to the proximal end of the microcatheter and connected to a continuous flush system. With the aid of a suitable guidewire, and using standard catheterization techniques and fluoroscopic guidance, the microcatheter was advanced up to and across the occlusion so that the distal end of the microcatheter is positioned distal of the occlusion. The guidewire was removed from the microcatheter and optionally contrast media was gently infused through the microcatheter to visualize the distal end of the occlusion. The insertion tool with the preloaded retrieval device 200 was then removed from the packaging hoop. The distal end of the insertion tool was inserted through the RHV of the microcatheter and then waited until fluid was seen exiting the proximal end of the insertion tool, confirming that device 200 was flushed. The insertion tool was then advanced until it contacted the hub of the microcatheter and the RHV was fully tightened to hold the insertion tool securely in position. The insertion tool was confirmed as being fully seated in the hub of the RHV before proceeding to advance device 200 until at least half of the shaft length of shaft 206 was inserted into the microcatheter, at which point the insertion tool was removed.

Regarding positioning and deployment, device 200 continued to be advanced towards the distal tip of the microcatheter (e.g., until the distal radiopaque tip 208 of the device 200 was aligned with the distal tip). Device 200 optionally included bands positioned on the proximal portion of shaft 206 to assist in minimizing the amount of fluoroscopic exposure required during insertion of device 200. If using a standard microcatheter (total length of 155 cm and a 7 cm RHV), then when the first band on the shaft 206 approached the RHV, while the tip of device 200 was approximately 8 cm from the distal end of the microcatheter. When the second band on the shaft 206 approached the RHV, the tip of device 200 was nearing the distal end of the microcatheter. Device 200 was then advanced in the microcatheter and positioned within the clot and left to embed for 3-5 minutes prior to withdrawal.

Device 200 was optionally supplied preloaded within an insertion tool. In such applications, the physician inserted the insertion tool into the hub of a pre-positioned microcatheter and advances the clot retrieval device forward out of the insertion tool and into the microcatheter.

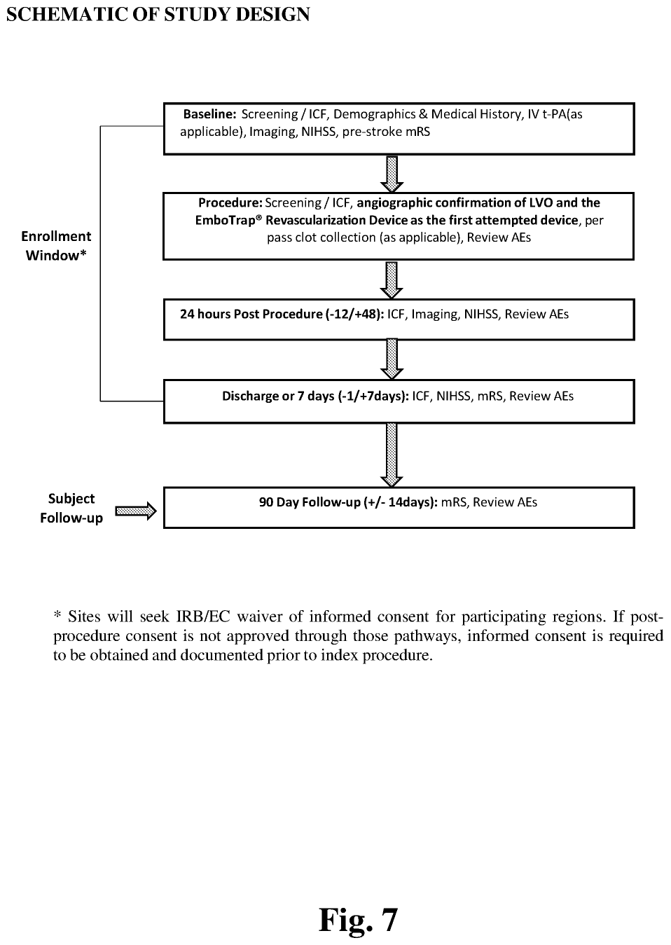

During each study, device 200 tested was used for at least the first attempt. If an additional passes or retrieval devices were used, it was noted. Any captured thrombus was carefully removed therefrom and analyzed. FIG. 7 of this disclosure shows a representative overview of the study flow using the patients of each study with the clot retrieval device 200.

Patients associated with each study included those with acute ischemic stroke with confirmed intracranial Large Vessel Occlusion treated with endovascular treatment using the stent retriever of this disclosure as the first line device across multiple different centers.

### Patient Selection

The present study was for “all comers” and had a minimum number of inclusion criteria. For each included patient, age, gender and cardiovascular risk factors (e.g., diabetes mellitus, obesity, smoking, high blood pressure, hyperlipidemia) were recorded. Typically, initial imaging was a brain CT with cervical and intracranial angiography or brain MRI with time of flight angiography, depending on hospital protocol. The ASPECT (Alberta Stroke Program Early CT) score was evaluated by experienced neuroradiologists on either modality, and the NIHSS score by neurologists. Patients were treated up to 24 hours from time of stroke onset or time last known well in case of wake-up stroke.

All patients with confirmed large-vessel occlusion were treated with the clot retrieval device 200 of this disclosure. The device 200 was used as first-line device and the number of attempts using the device 200 were also left to the discretion of the treating physician

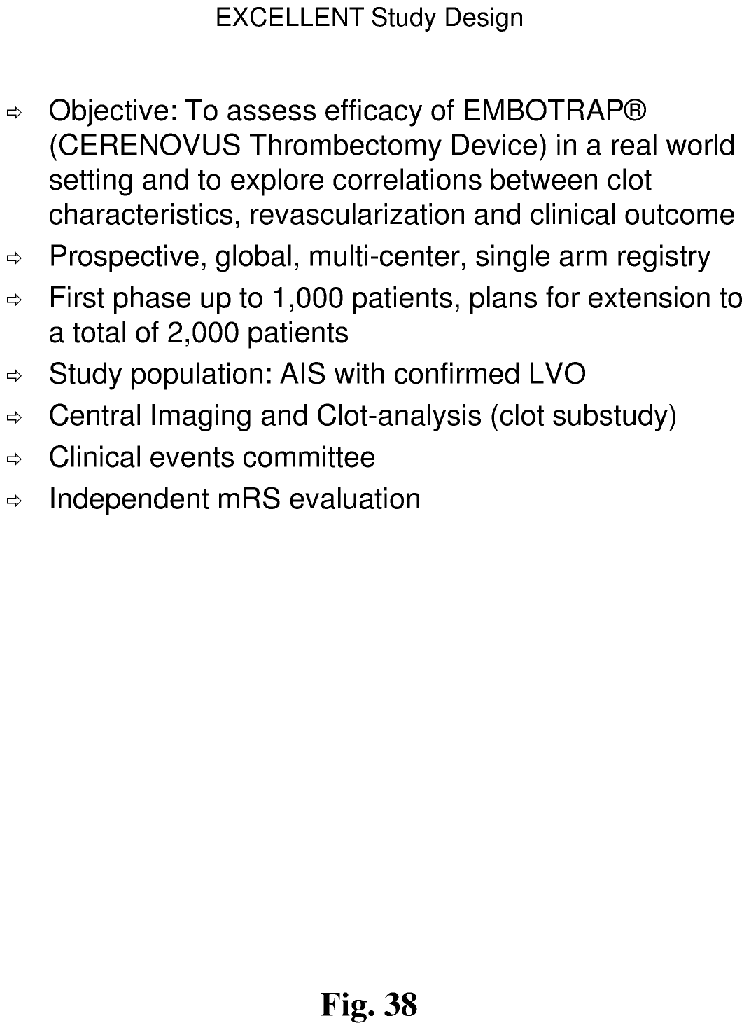

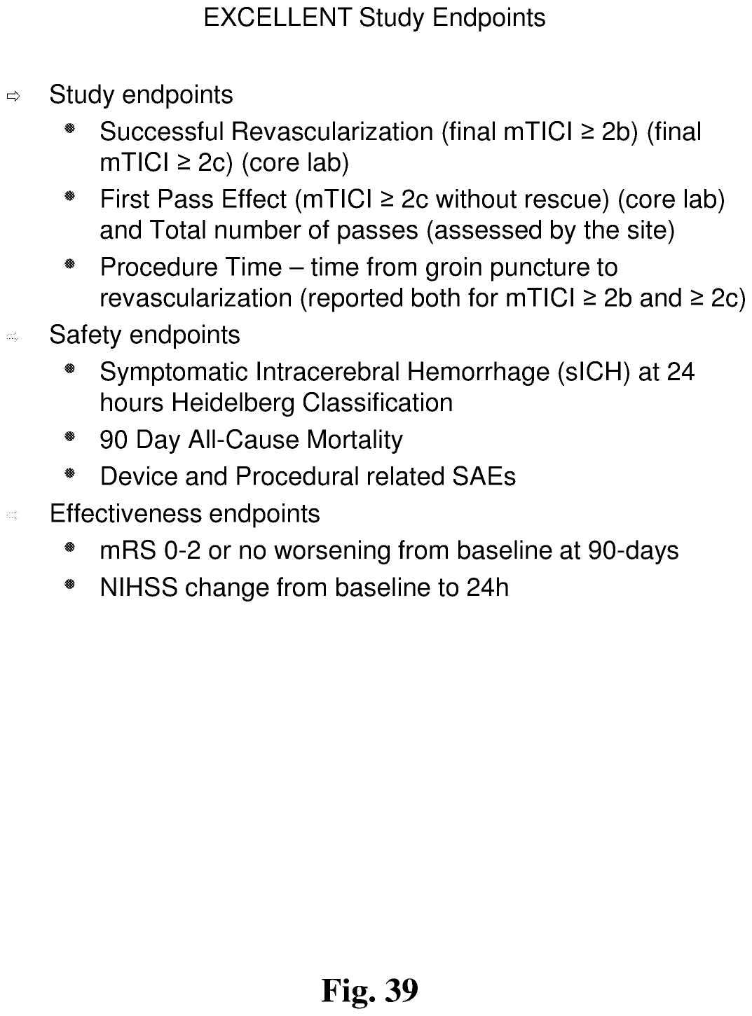

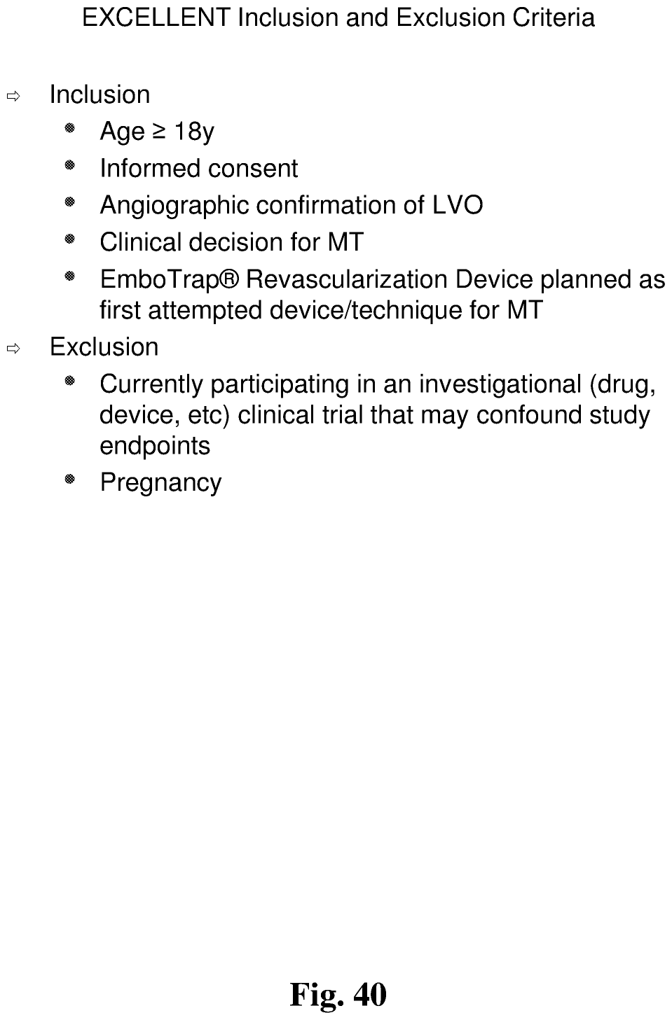

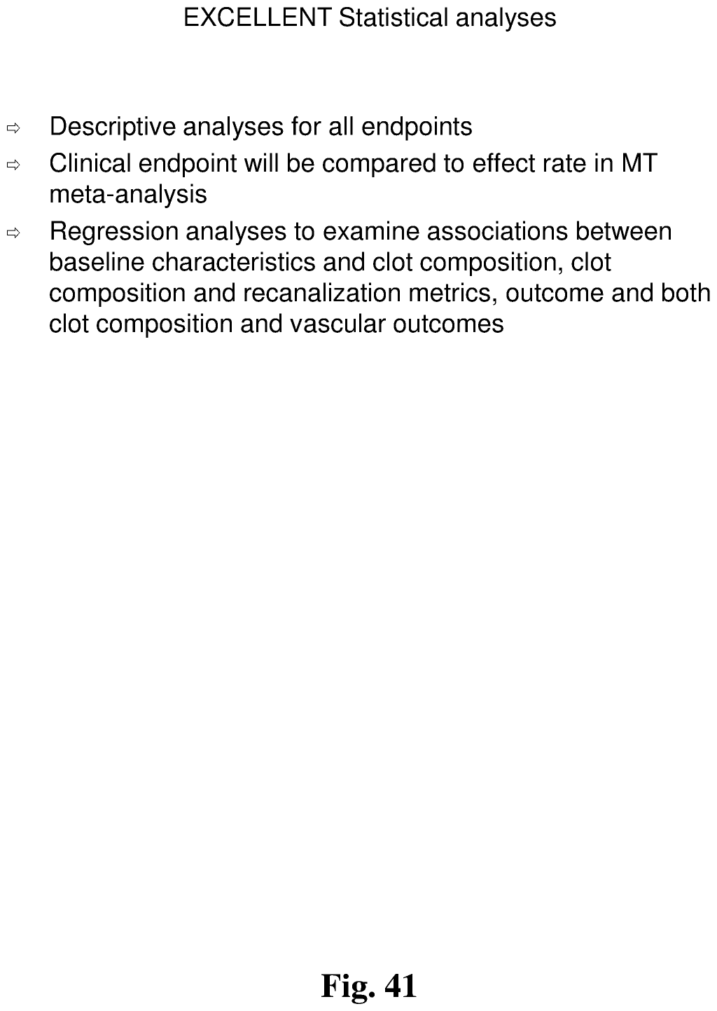

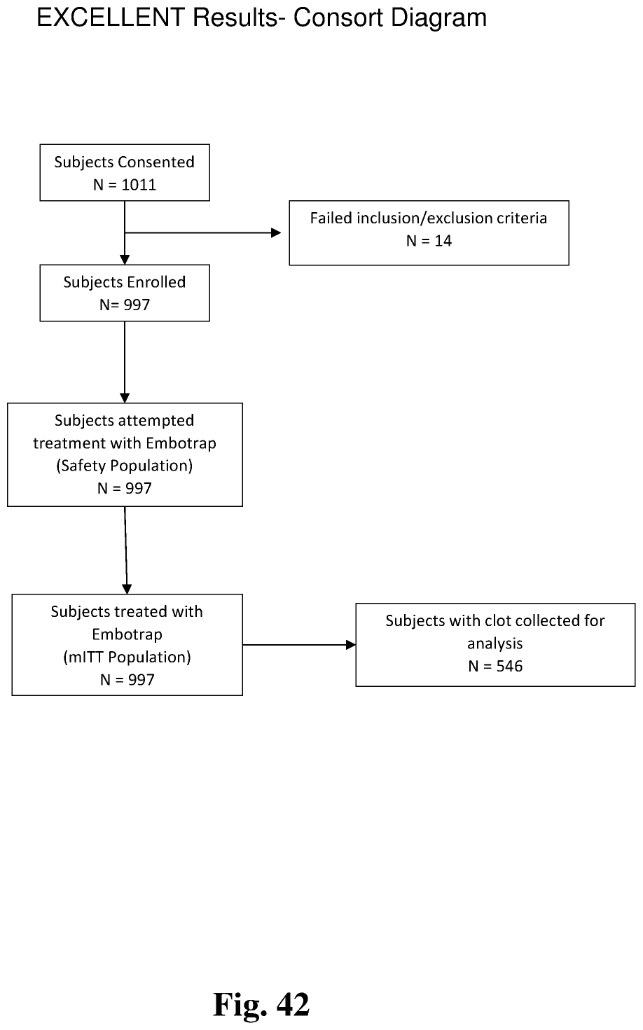

The study objective was to assess the efficacy of device 200 in a real-world setting, as well as to explore correlations between patient comorbidities, clot characteristics, revascularization rates, and clinical outcomes. Patients were treated with device 200 and angiographic imaging was collected per pass for subsequent review by an independent lab. Clot was collected from sites who have the infrastructure to support clot collection after each pass of the device for follow on analysis by an independent lab. In addition, adverse events (AE) were collected throughout the entire follow-up period of the patient except where otherwise noted, regardless if they were related to the device 200. See FIGS. 38-42.

An adverse event (AE) is defined as any untoward medical occurrence, unintended disease or injury, or untoward clinical signs (including abnormal laboratory findings) in patients, users or other persons, whether or not related to the investigational medical device. Adverse Events (AE) of interest are the events listed below. In addition, additional adverse event data was collected by the physician at their discretion to capture notable adverse events or serious adverse events not included in the list of events of interest below and to capture unanticipated adverse events. Adverse Effects were All Strokes, Device-Related AEs, Procedure-Related AEs, Vessel Perforation, Dissection or Injury, Vascular Access Injury, Neurologic Deterioration (change in NIHSS ≥4pts from last known) through 7 days/discharge; whichever comes first, and Symptomatic ICH through 24 hr imaging. In addition, all mortality was reported in the study regardless of causality, ICH, and Emboli in New Territory. It is known to those of skill in the art that the term “rate” is intended to refer to the rate for a particular population of patients according to a particularly clinical investigation rather than information or levels related to a single patient. However, herein the term “rate” and “level” can be used interchangeably. In any study, single patient levels are used to determine “rates”. Any one patient's level may be the notable point in a reported rate.

A serious adverse event (SAE) is defined as an adverse event that Led to death, Led to serious, deterioration in the health of the patient, that either resulted in a life-threatening illness or injury, or a permanent impairment of a body structure or a body function, or in-patient or prolonged hospitalization, or medical or surgical intervention to prevent life-threatening illness or injury or permanent impairment to a body structure or a body function, Led to fetal distress, fetal death or a congenital abnormality or birth defect. An adverse device effect is defined as an adverse event related to the use of device 200. A serious adverse device effect is an adverse device effect that has resulted in any of the consequences characteristic of an SAE. An Unanticipated Serious Adverse Device Effect (SADE) is a serious adverse device effect which by its nature, incidence, severity or outcome has not been identified in the study parameters. Unanticipated Adverse Device Effect (UADE) effect is any serious adverse effect on health or safety or any life-threatening problem or death caused by, or associated with, a device, if that effect, problem, or death was not previously identified in nature, severity, or degree of incidence in the study.

Neurologic evaluation in each study was performed through repeat NIHSS determinations in line with standard of care at 24 hours (−8/+12 hours) and at 7 days (or discharge whichever is sooner) time points post-procedure. An additional NIHSS score was obtained when any signs of neurologic deterioration occur or in the event of an ICH to assess the degree of deterioration. A certified examiner performed all neurologic evaluations and the 90-day evaluation was used to record the mRS score.

Inclusion criteria for each study included the patient was aged greater than or equal to 18 years and angiographic confirmation of an occlusion of a Large Vessel Occlusion (LVO). Further, the patient was indicated for neurothrombectomy treatment by the interventionalist and device 200 was chosen as the first line device. The exclusion criteria for each study included females who were pregnant and/or the patient was participating in another study involving an investigational device or drug.

### Results of the Study

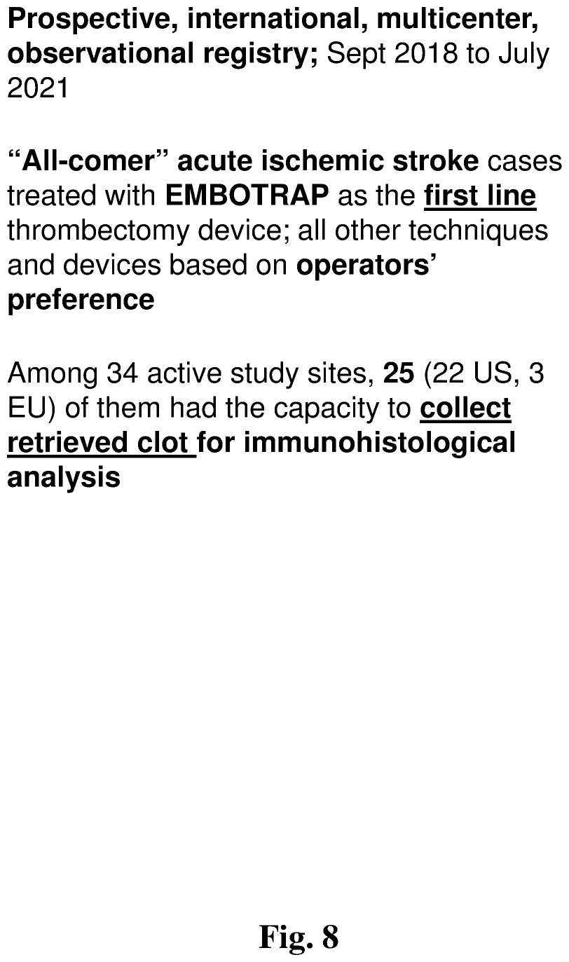

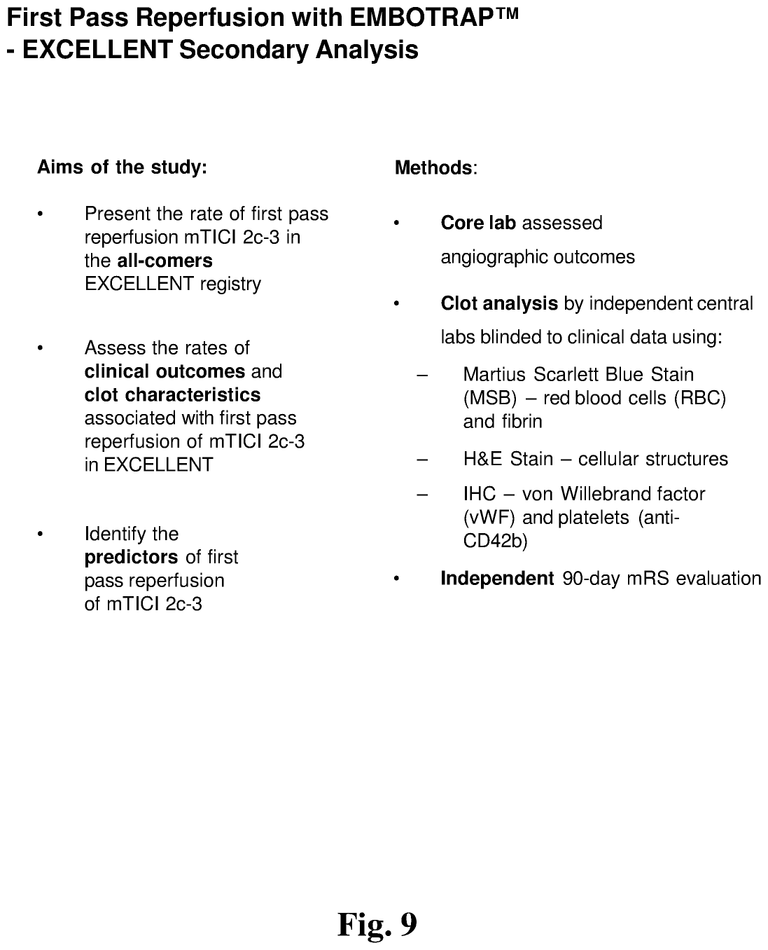

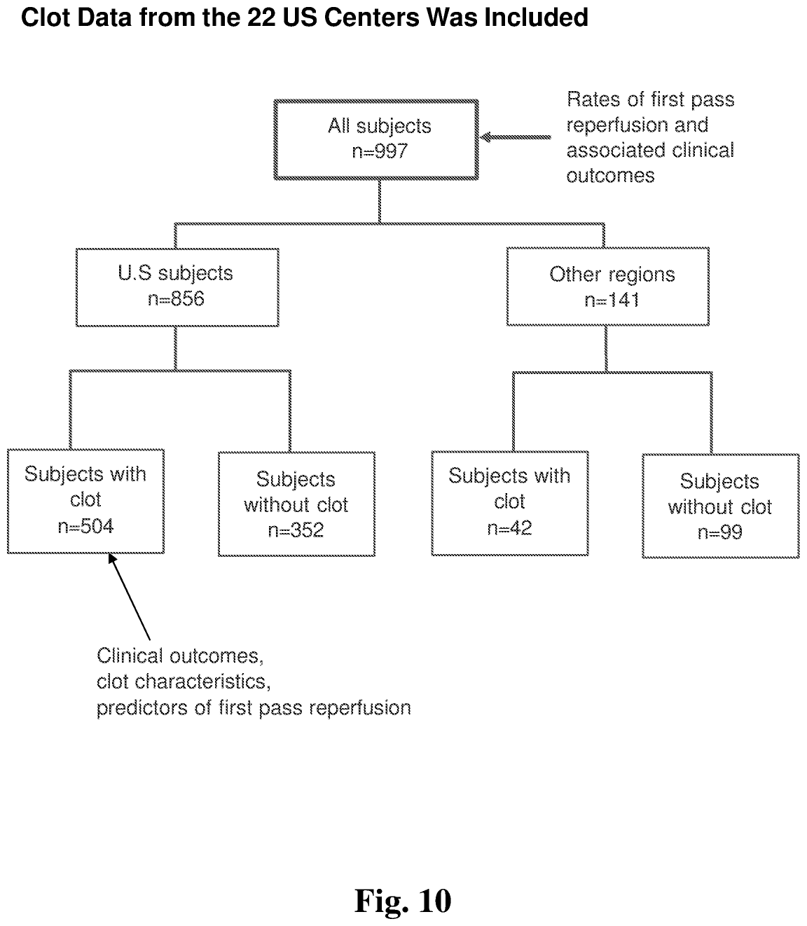

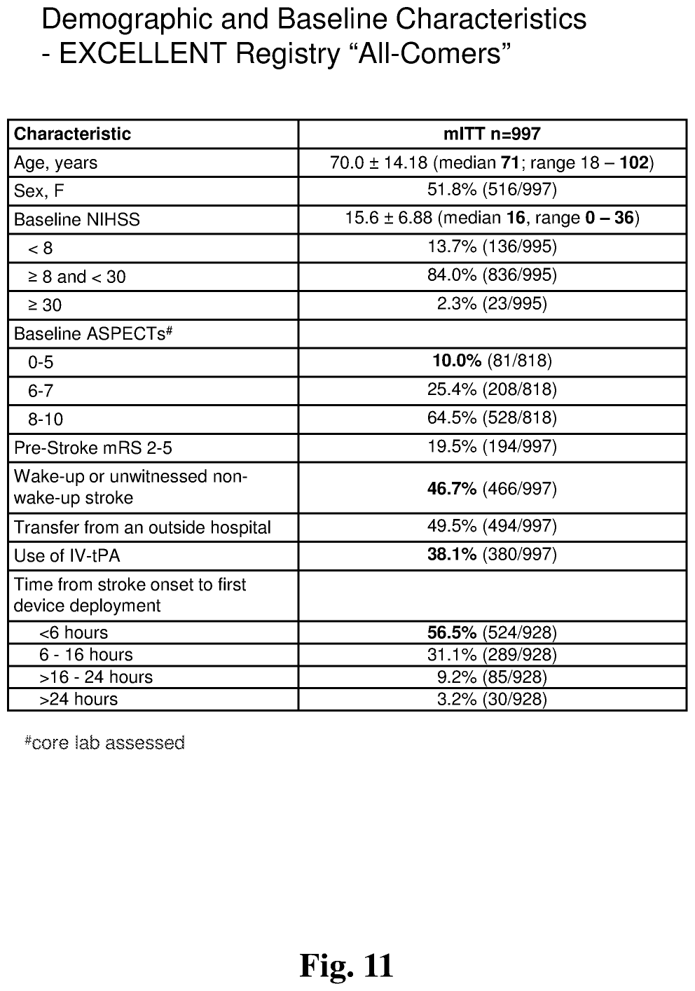

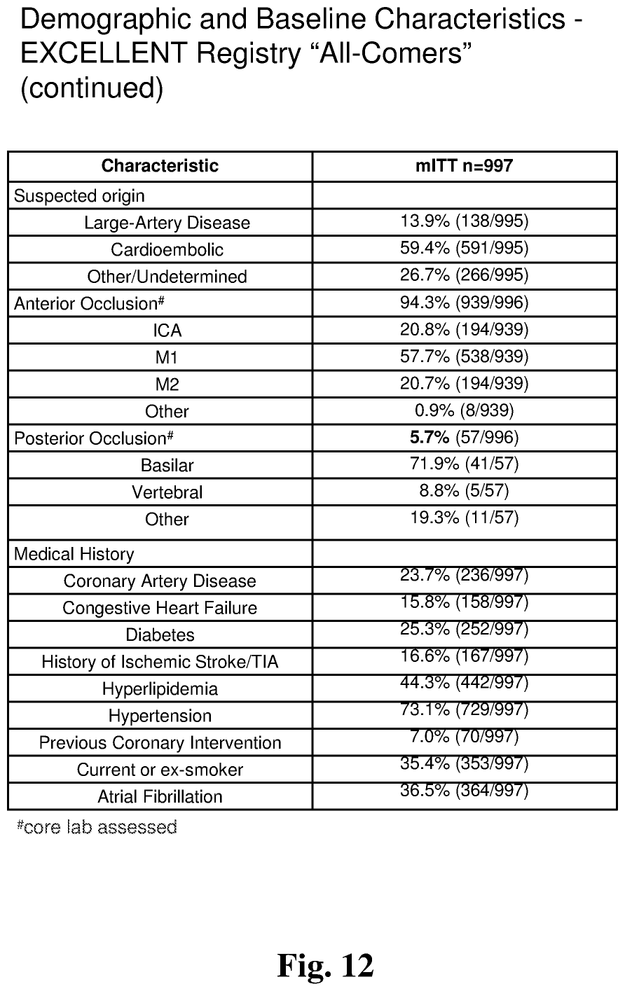

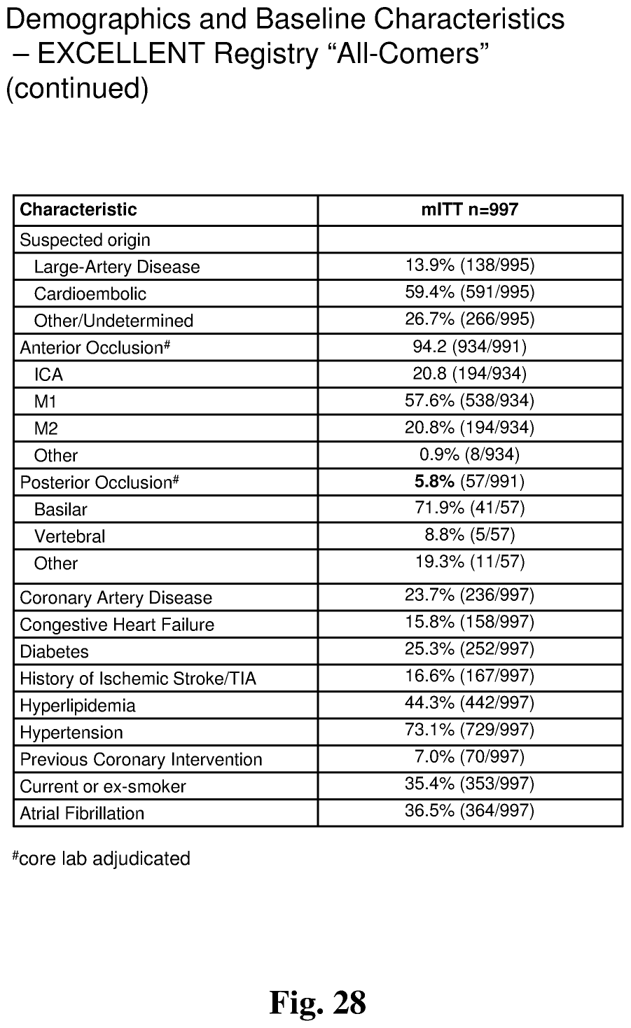

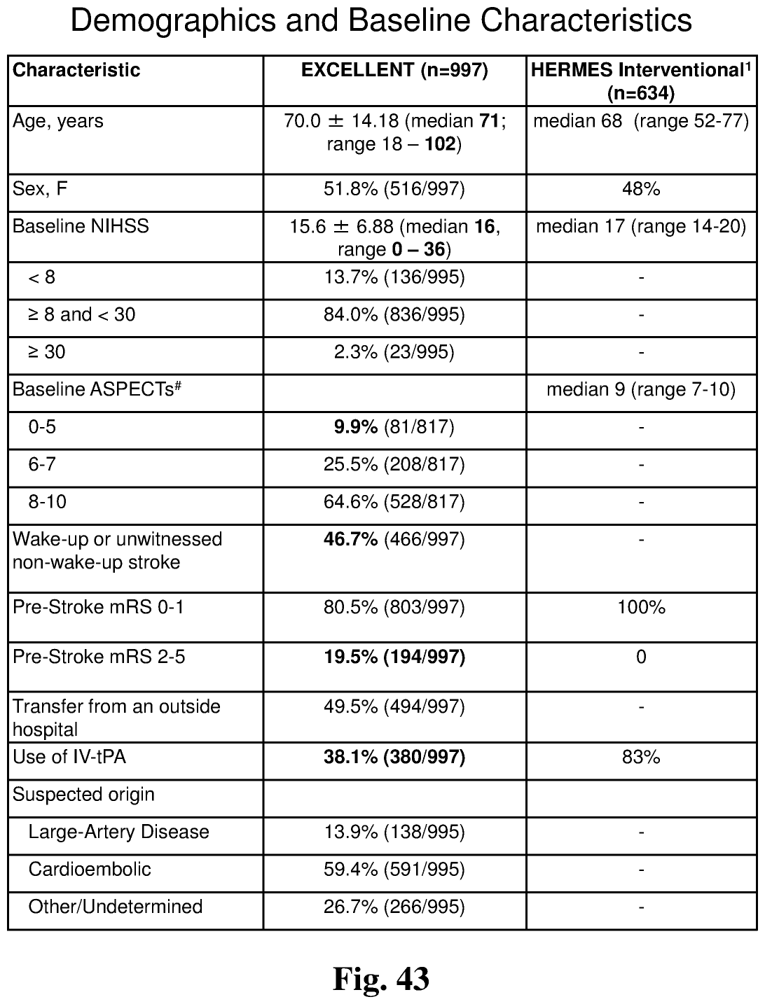

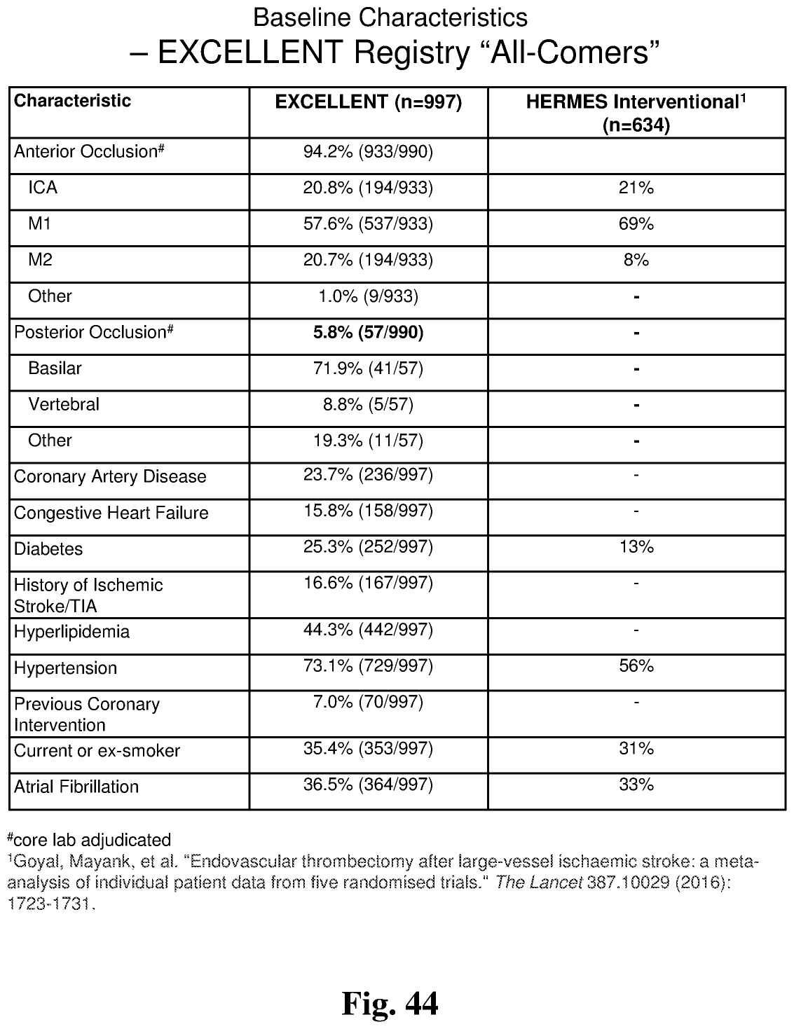

In the study, 997 patients from 22 facilities in the United States (481 men and 516 women; median age 71 years; range, 18-102 years) were treated with device 200 from September 2018 to July 2021 as outlined in FIGS. 8-10. Baseline characteristics of the study are also summarized in FIGS. 11 and 43 while intra-procedural characteristics are summarized in FIGS. 12, 28, and 44. Median NIHSS at admission was 16 (range 0-36) and the baseline ASPECT score varied over the range, where 10% of the patients scored 0-5, 25.4% scored 6-7, and 64.5% scored 8-10. Three hundred and eighty patients (38.1%) received IV tPA. The time from symptom onset to device 200 deployment was divided into ranges 56.5% received deployment in less than 6 hours, 31.1% between 6 and 16 hours, 9.2% from greater than 16 hours to 24 hours, and 3.2% at greater than 24 hours. The median time from groin puncture to first mTICI ≥2b was 29 minutes (37.8 min ±28.14). The number of thrombectomy attempts ranged from 2.1±1.45with a median of 1 (range 1-10). The number of patients with less than or equal to three passes to mTICI ≥2b was 85.1% (848/997).

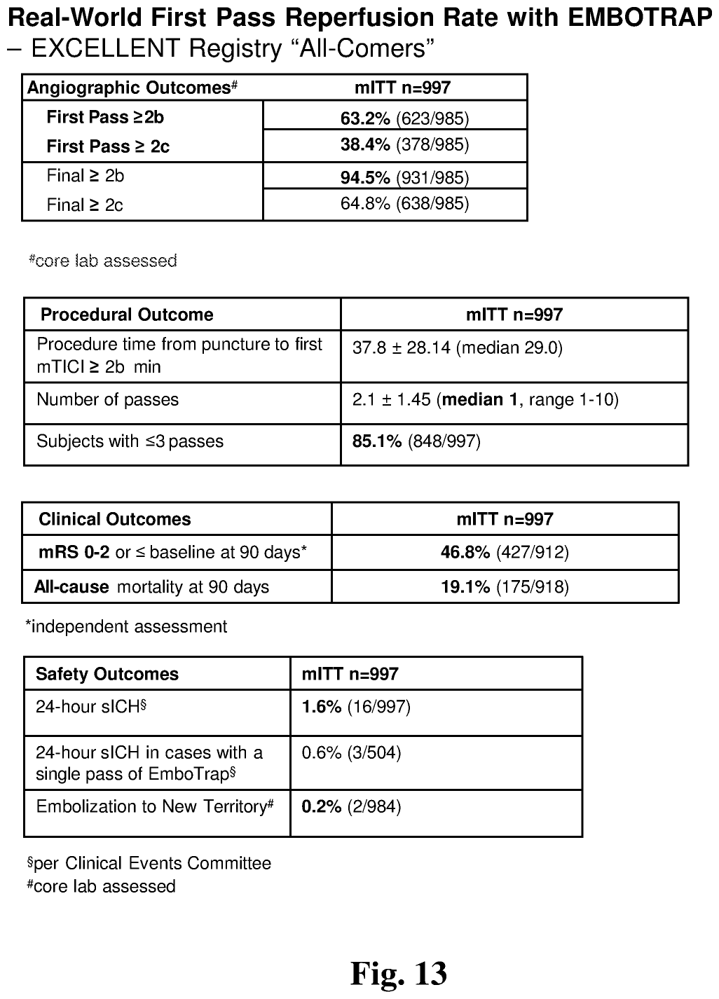

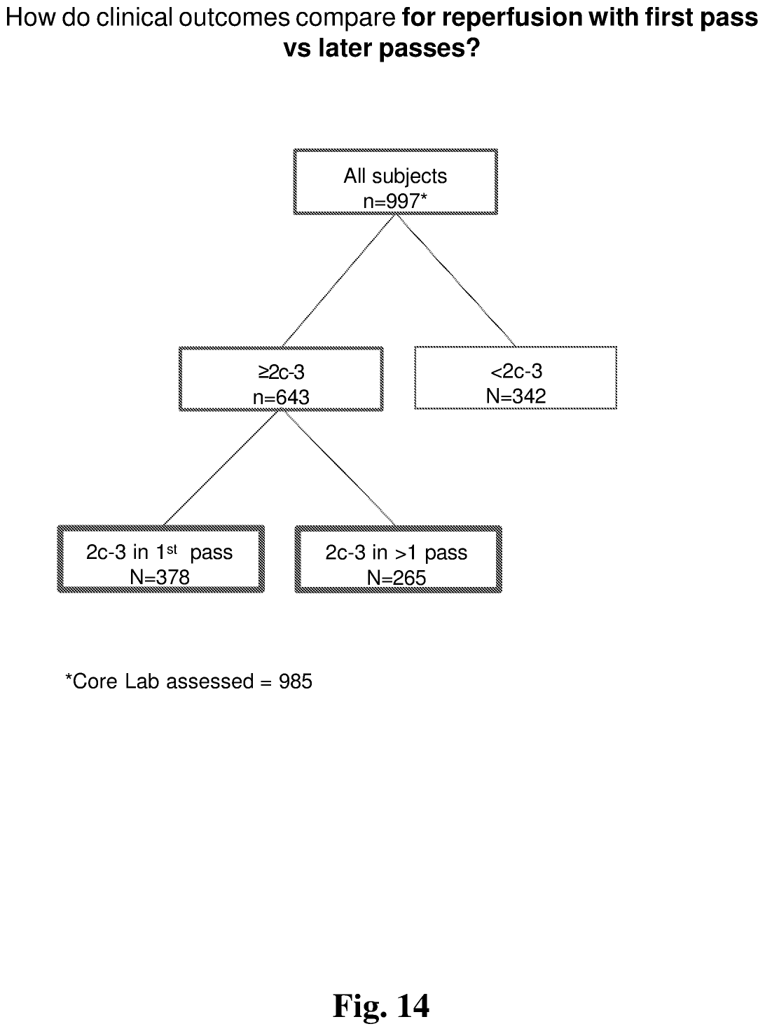

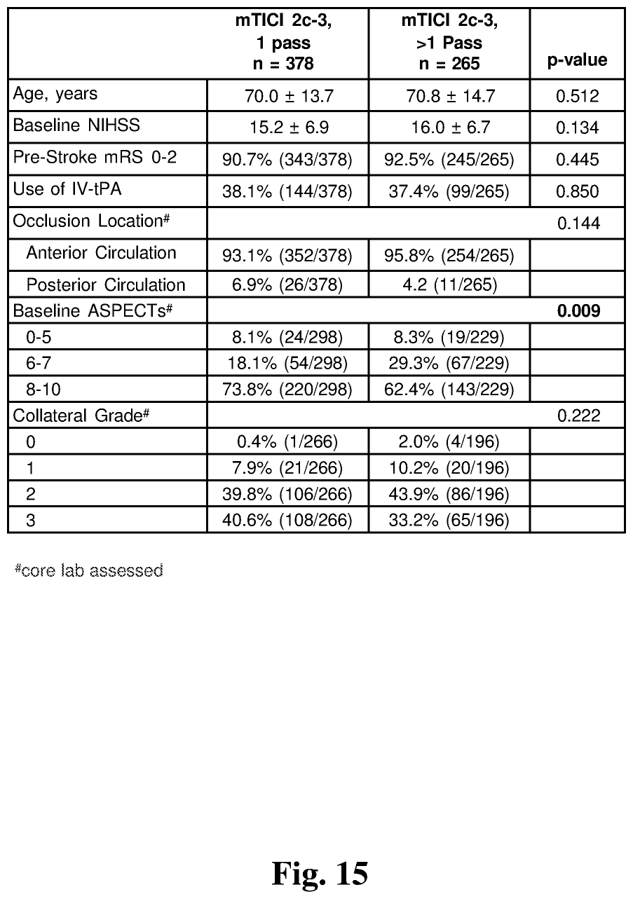

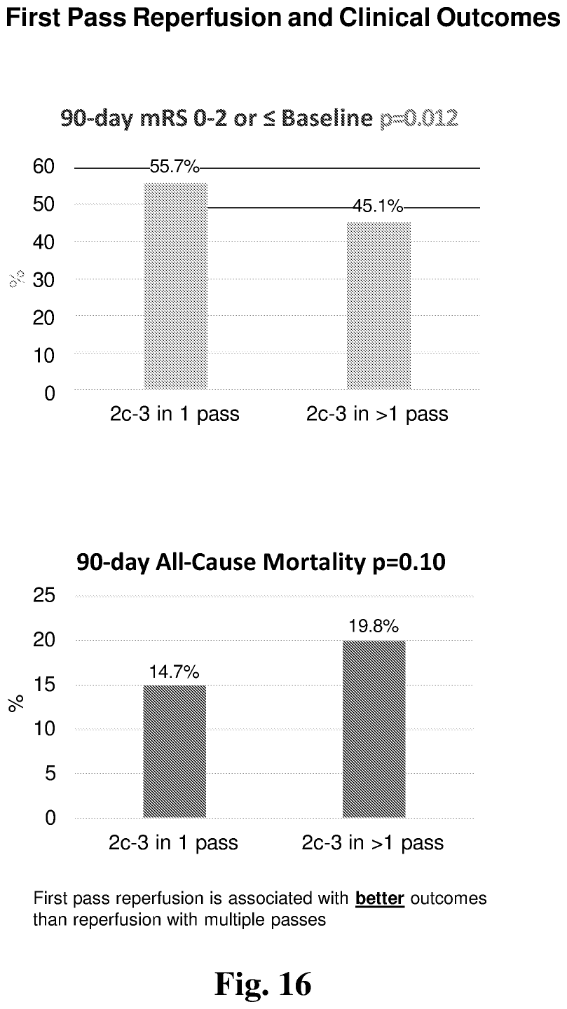

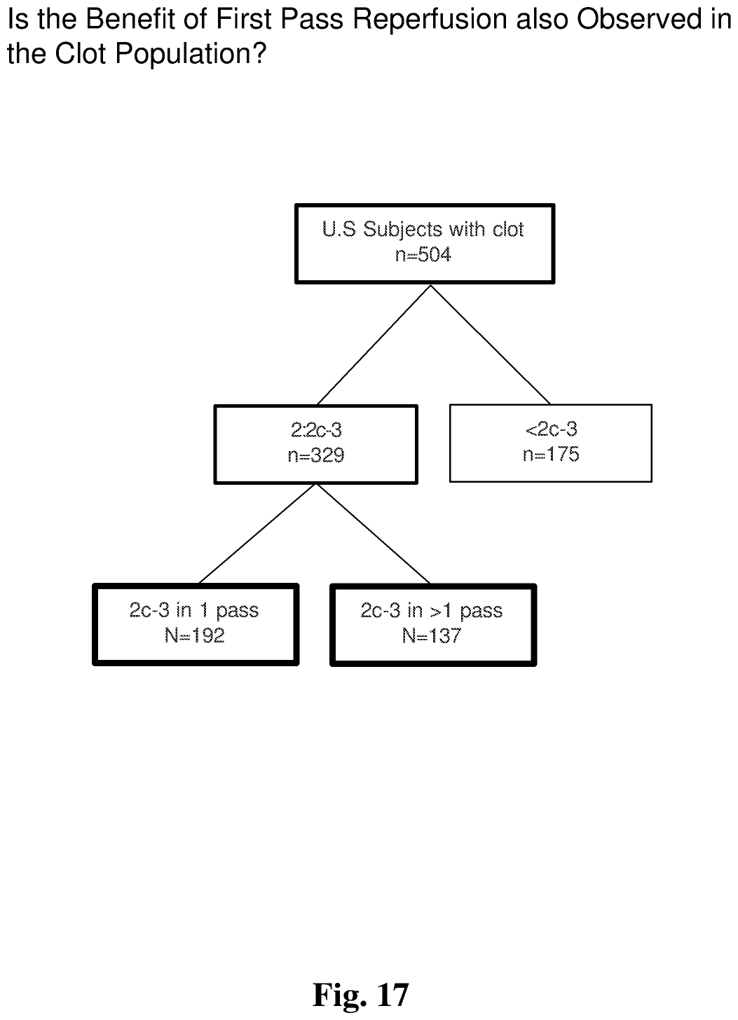

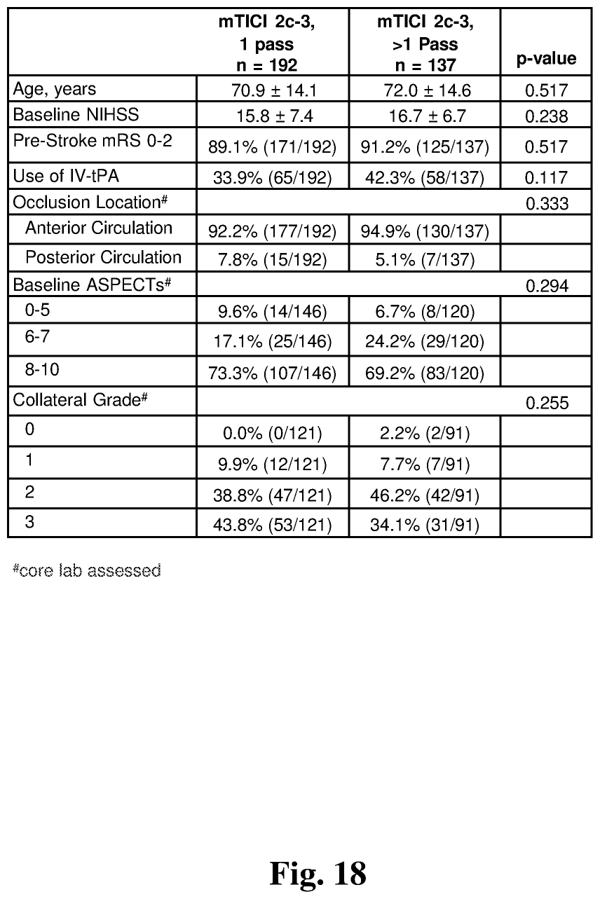

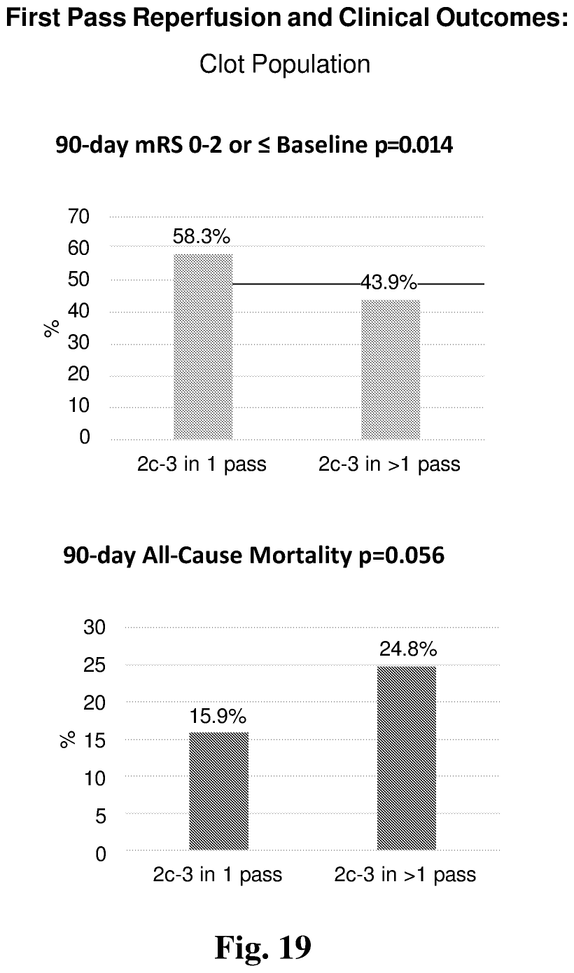

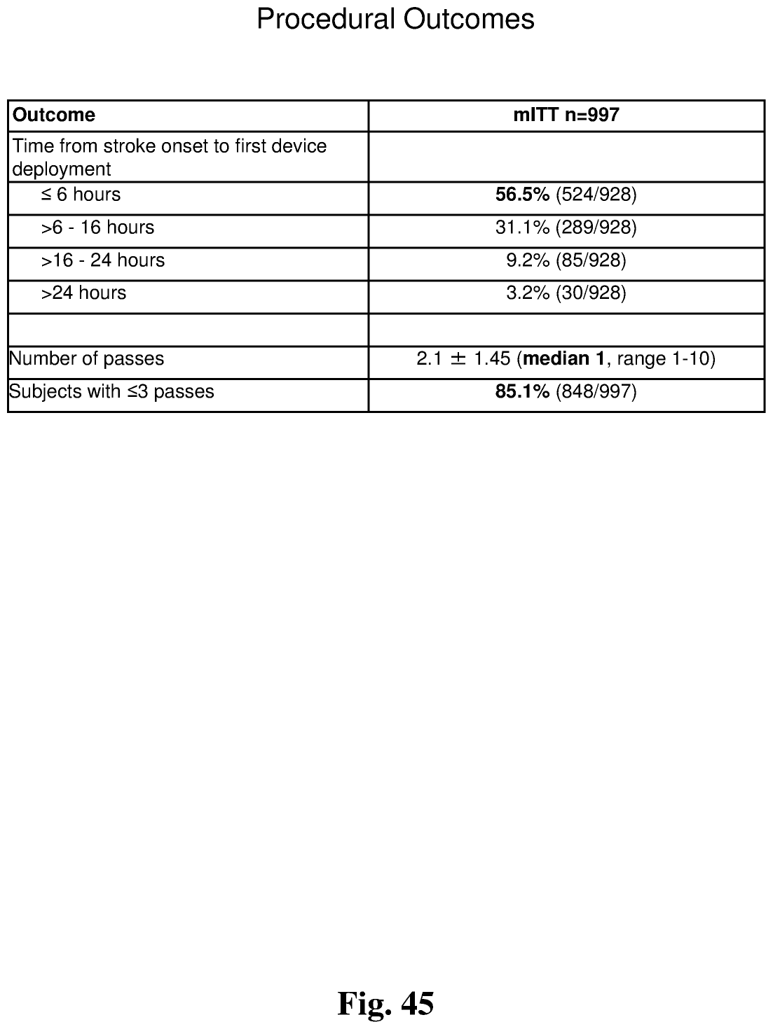

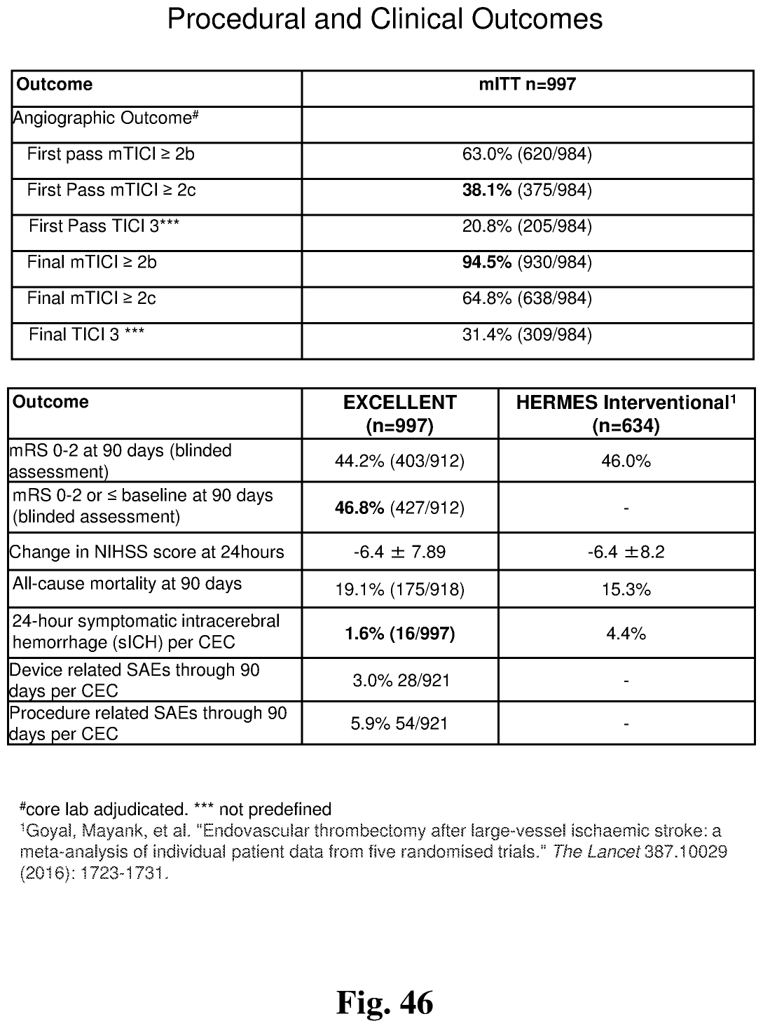

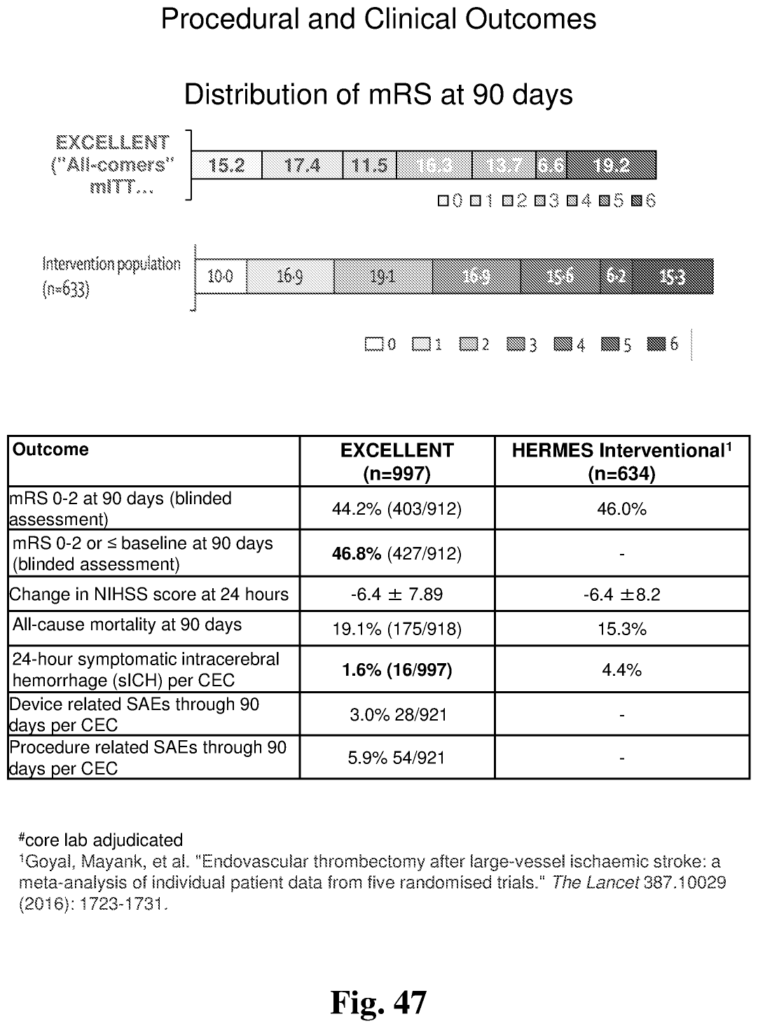

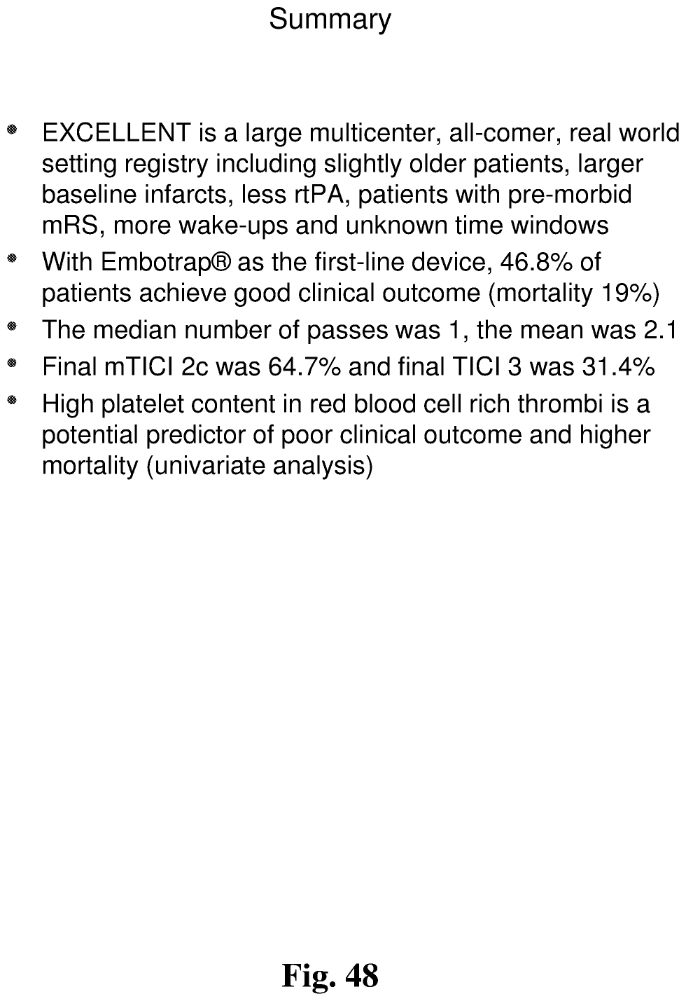

Device 200 in the study was used as a first line device Outcomes of the study are summarized in FIGS. 13-19, whereby 63.2% (623/985) of the patients achieved an mTICI ≥2b on the first pass, with 38.4% (378/985) of the patients achieved an mTICI ≥2c on the first pass. 94.5% (931/985) of the patients achieved a final mTICI ≥2b and 64.8% (638/985) of the patients achieved a final mTICI ≥2c. General procedural outcomes are shown in FIGS. 45-47 and FIG. 48 provides a summary of the results.

FIG. 13 also provides both clinical and safety outcomes for certain AE. The clinical outcomes were reviewed over the population of 997 patients. An mRS of 0-2 or ≤baseline at 90 days was achieved in 46.8% (427/912) of the patients while 19.1% (175/918) of the patients experienced all-cause mortality at 90 days. The safety outcomes were likewise reviewed over the entire 997 patient population. A Symptomatic Intracerebral Hemorrhage (sICH) occurred within 24-hours in 1.6% (16/997) of the patients. While a 24-hour sICH in cases with a single pass of device 200 only occurred in 0.6% (3/504) of the patients. An embolization in New Territory occurred in 0.2% (2/984) of the patients.

The study also assessed the rates of clinical outcomes and clot characteristics associated with first pass reperfusion of mTICI 2c-3 with the device 200. As noted, the removed clots were saved and analyzed by a lab. The composition of clot components evaluated including Red Blood Cells (RBC), White Blood Cells (WBC), platelets, fibrin and other fibrous proteins. An analysis was performed to discover the relationships between histopathological findings and clinical factors including patient comorbidities, revascularization rates and clinical outcomes. The following parameters of the clot were measured/analyzed, the histology of the retrieved clot (including the red blood cell and fibrin content) measured using MSB (Martius Scarlett Blue) stain and H&E (Hematoxylin and Eosin) stain. The immunohistochemistry was also studied to determine Platelets and von Willebrand Factor and the geometry of clot by measuring total weight and clot area.

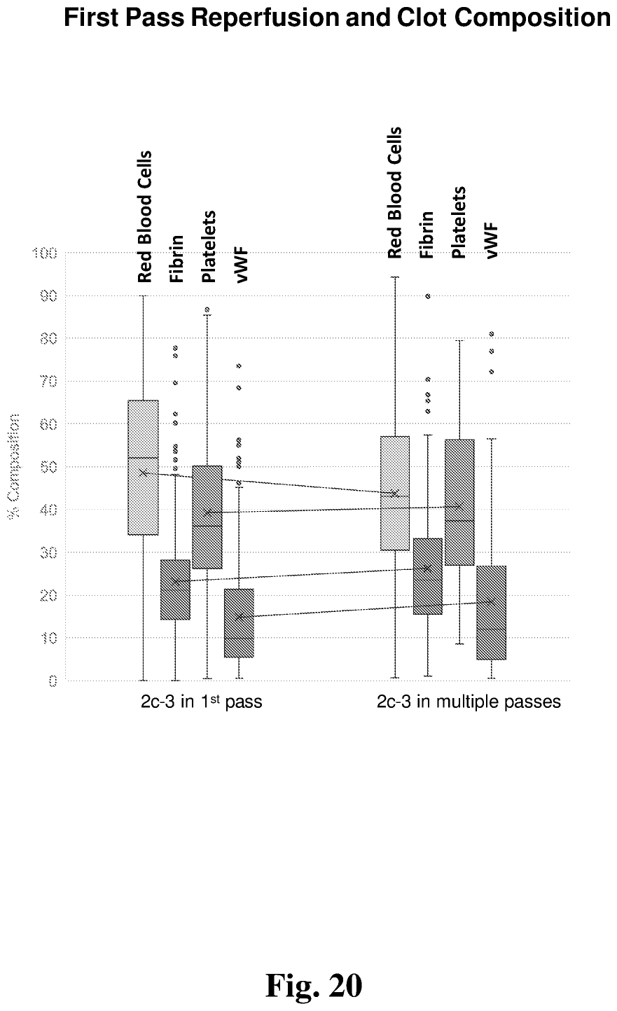

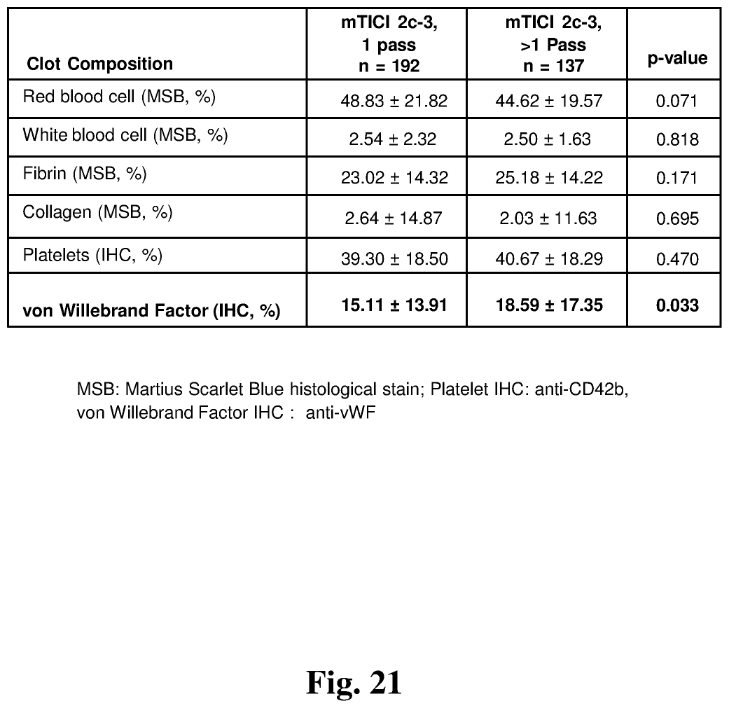

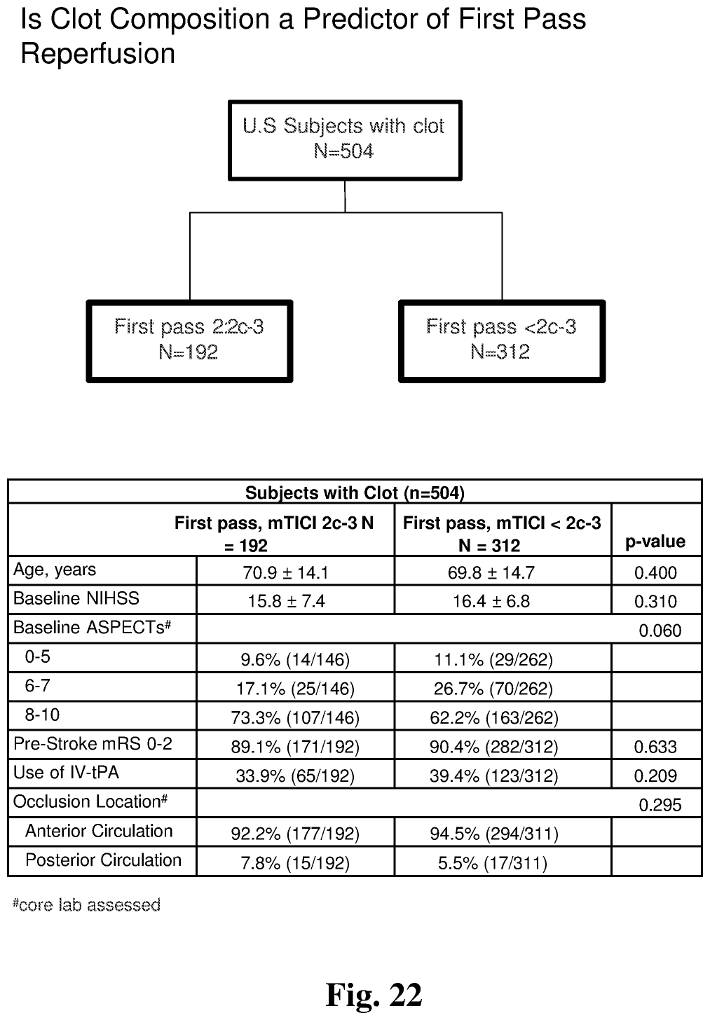

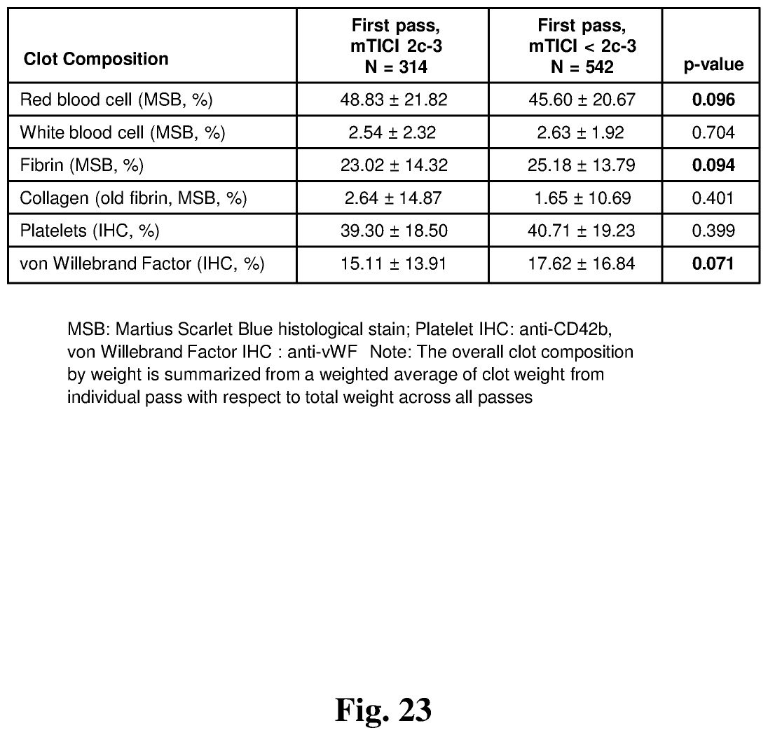

FIGS. 20-23 outlines the clot analysis and some significance to high mTICI scores in the first pass using device 200. The percentage of von Willebrand Factor (vWF) % in the clot seems to have a correlation to increased one-pass retrieval. The vWF is a blood glycoprotein that binds to other proteins to help platelet adhesion (e.g., blood clotting). One hundred and ninety-two patients had a 1-pass mTICI score of 2c-3 when their vWF % was between a percentage of 1.20 to 29.02 (average 15.11% ±13.91%). The greater than 1-pass with an mTICI score of 2c-3, for one hundred and thirty-seven patients who had a vWF % average of 18.59% ±17.35. Further, a first pass mTICI score of less than 2c-3, in five hundred and forty-two patients had a vWF % of 17.62 ±16.84.

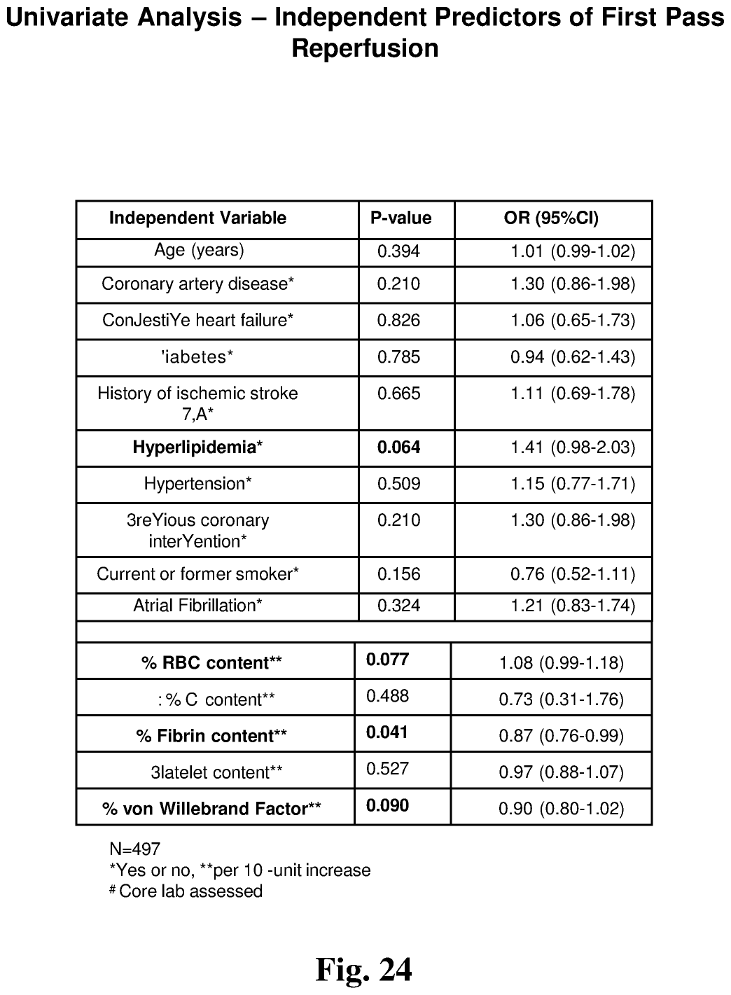

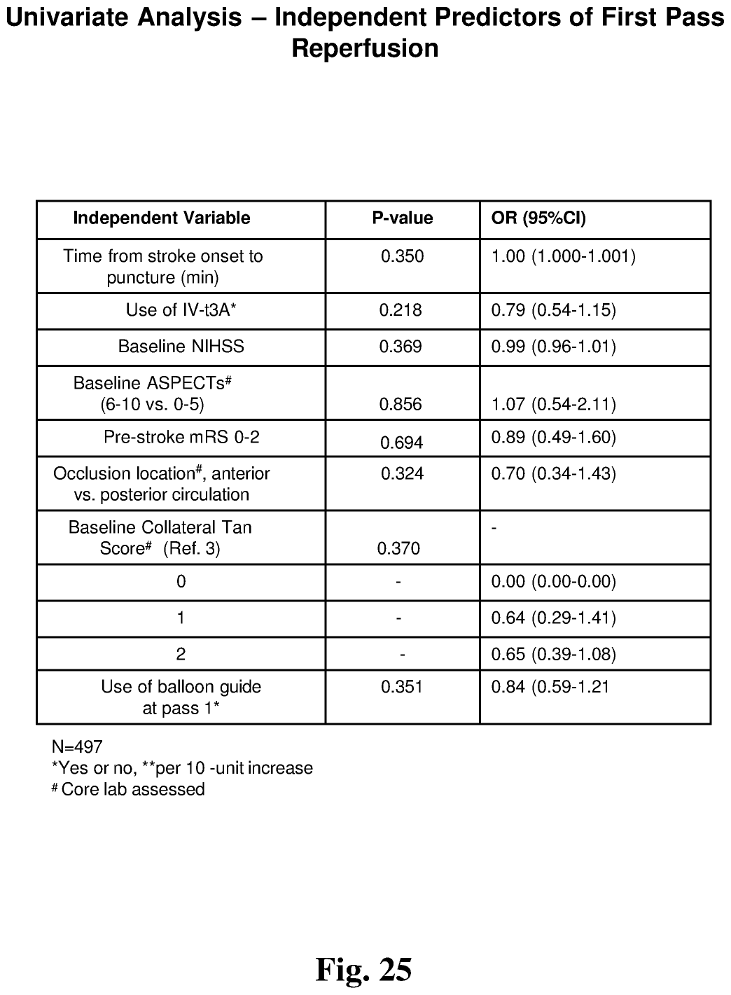

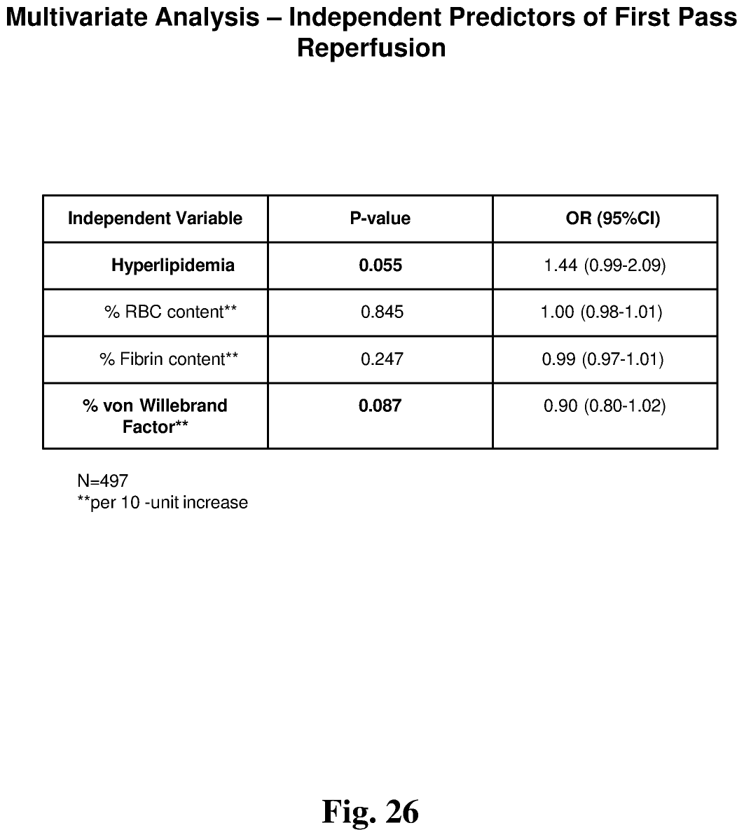

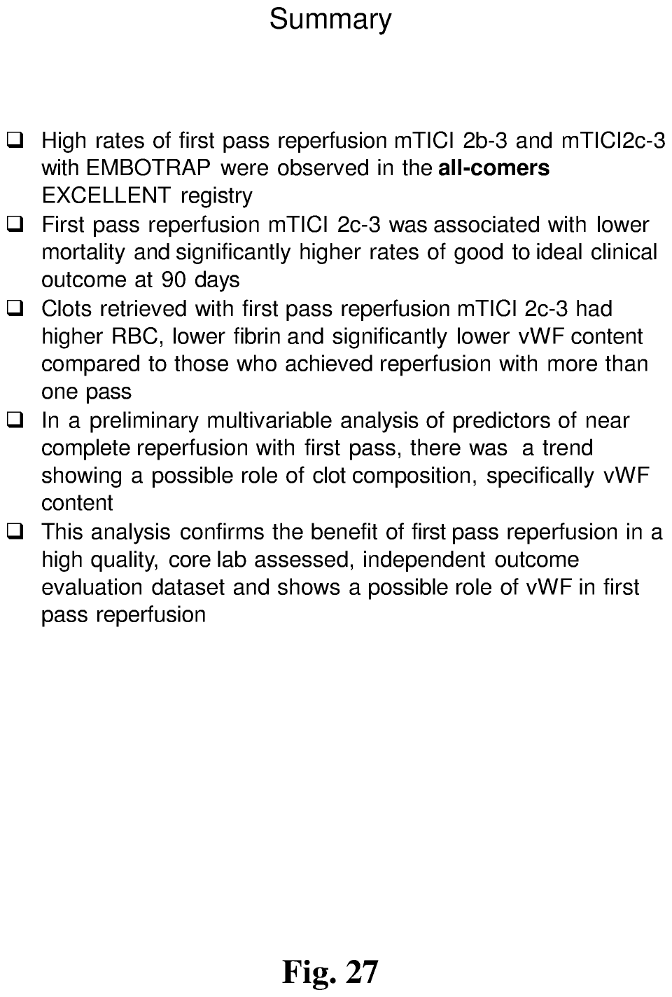

The study found that clots retrieved with first pass reperfusion mTICI 2c-3 had higher RBC, lower fibrin and significantly lower vWF content compared to those who achieved reperfusion with more than one pass (FIG. 27). In a preliminary multivariable analysis of predictors of near complete reperfusion with first pass, there was a trend showing a possible role of clot composition, specifically vWF content. Thus, there is a possible role of vWF in first pass reperfusion (FIGS. 24-27).

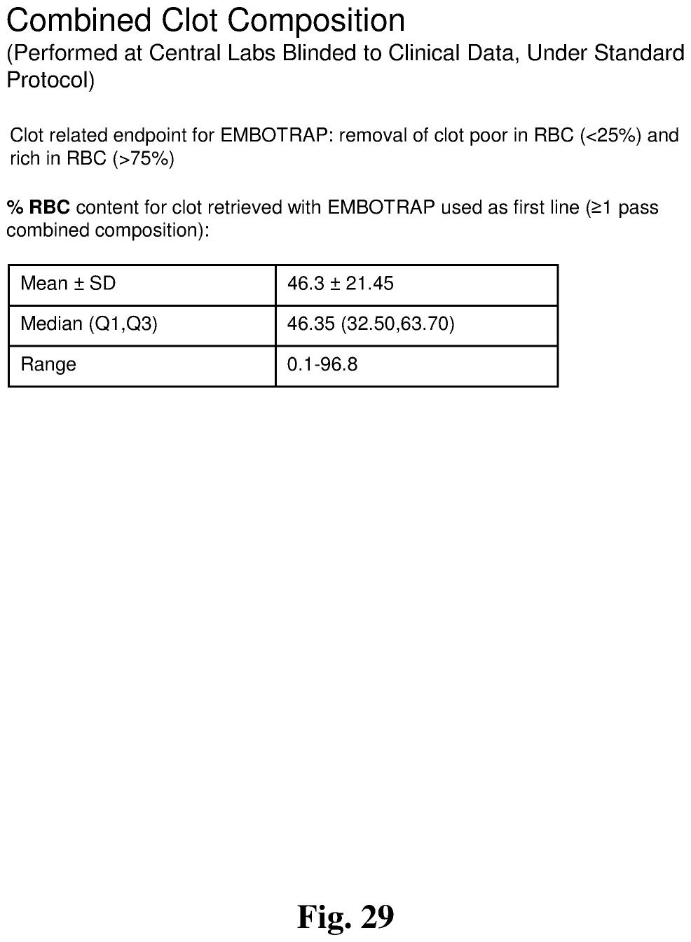

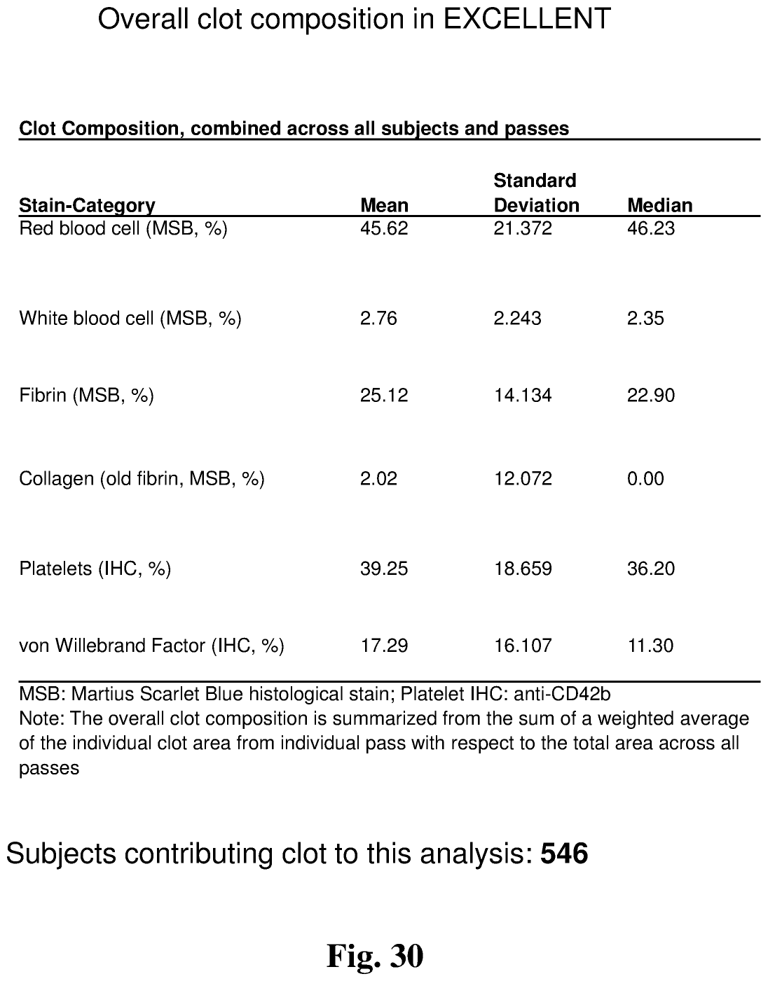

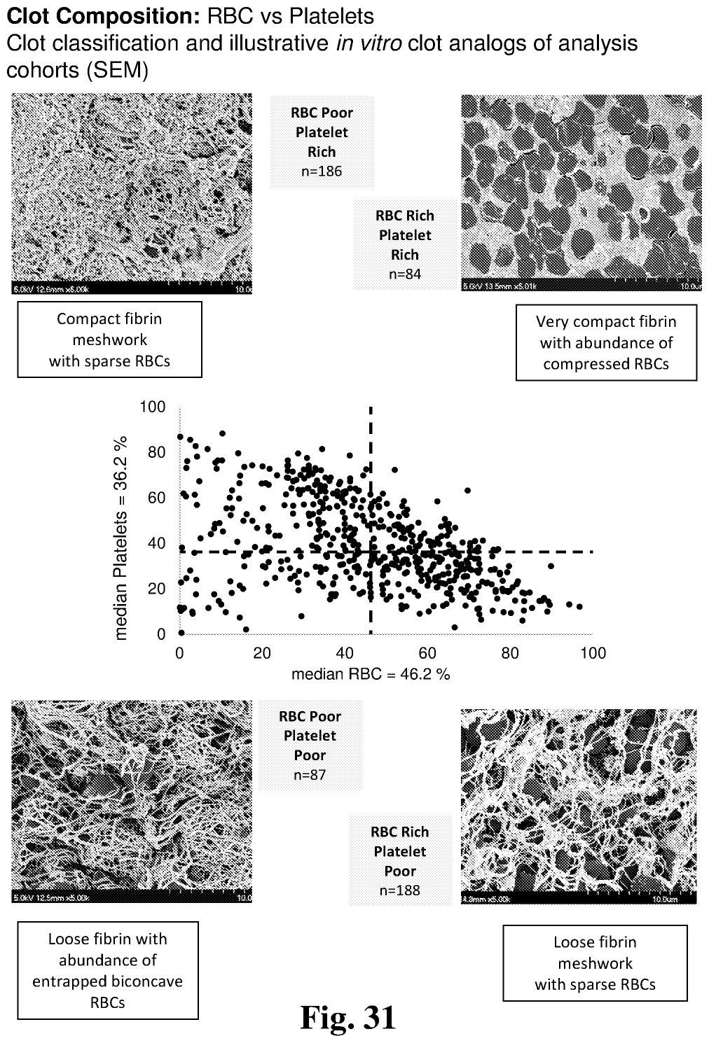

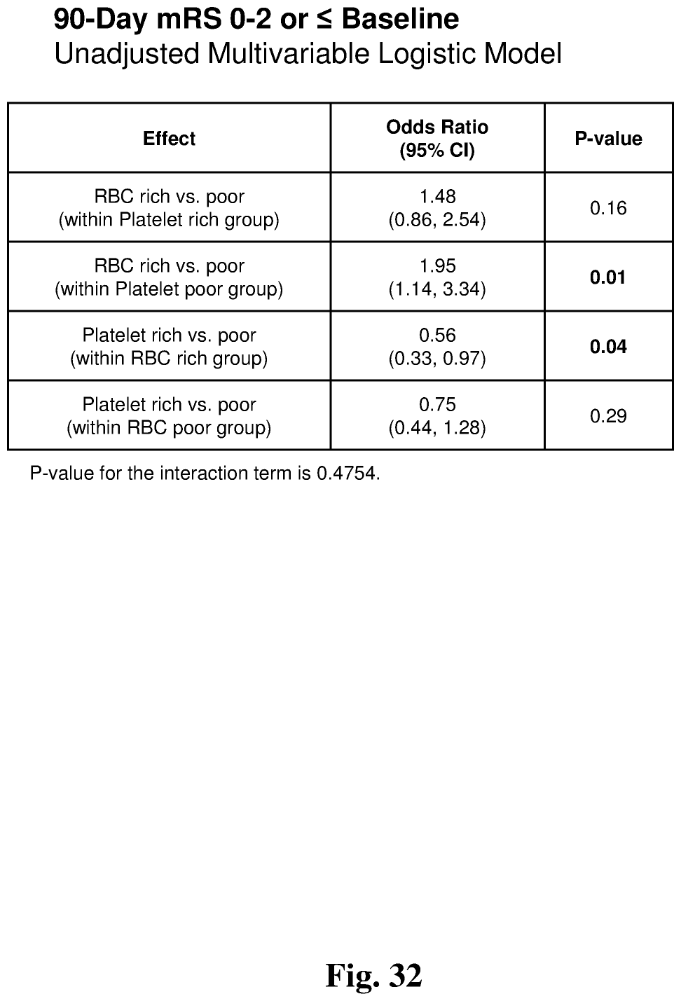

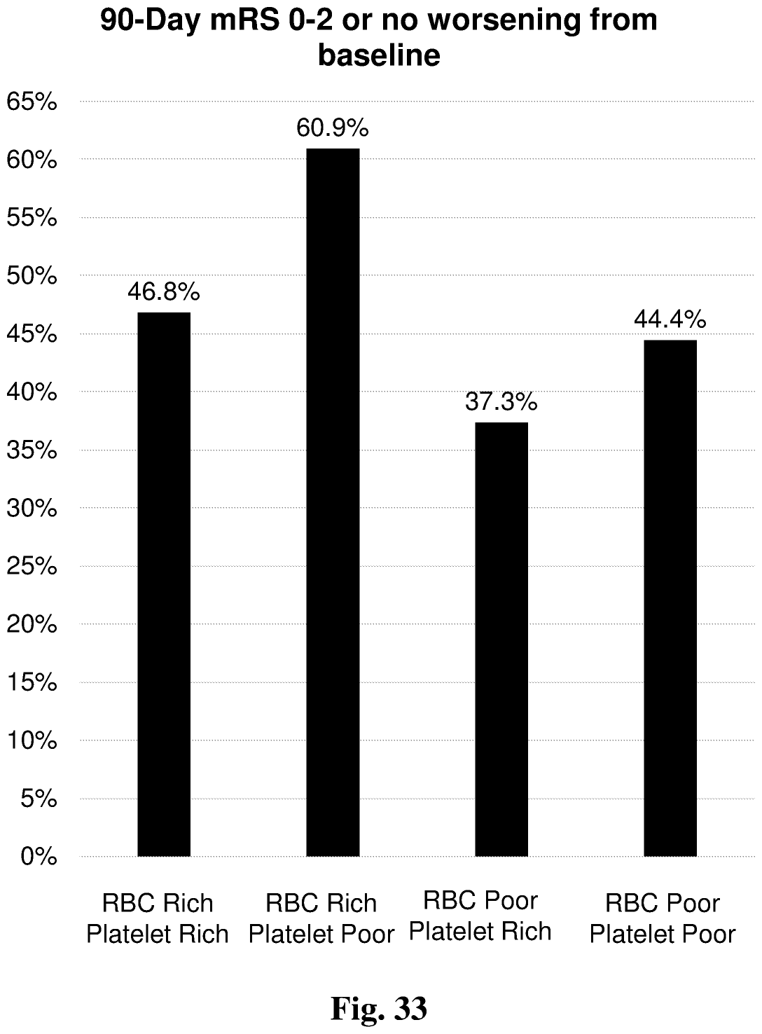

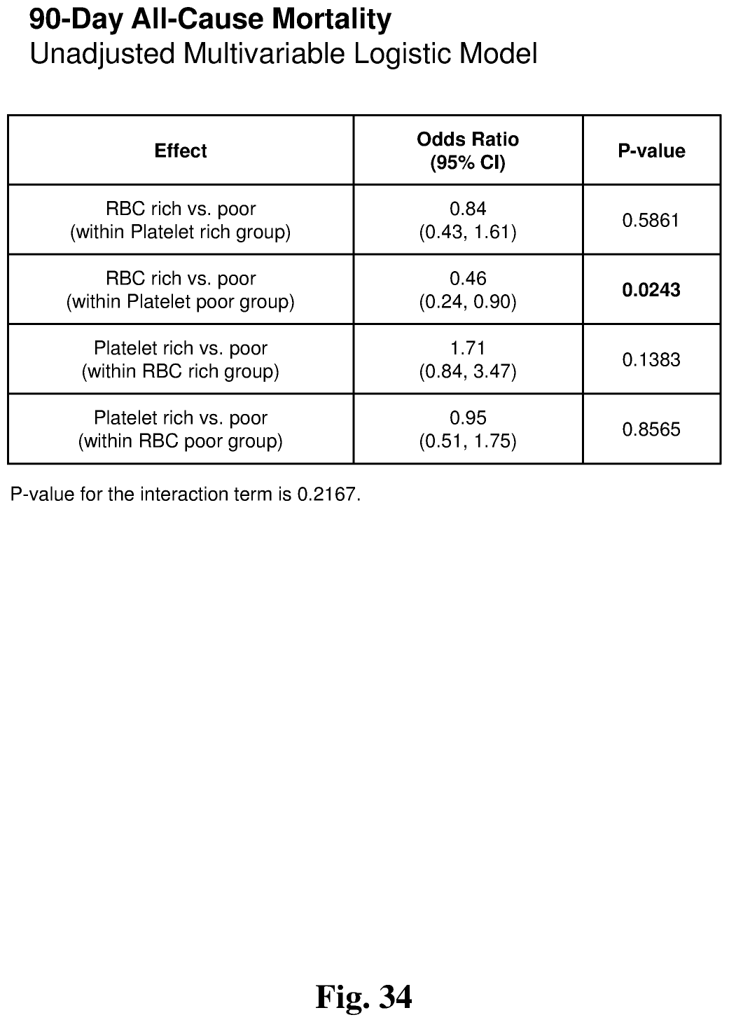

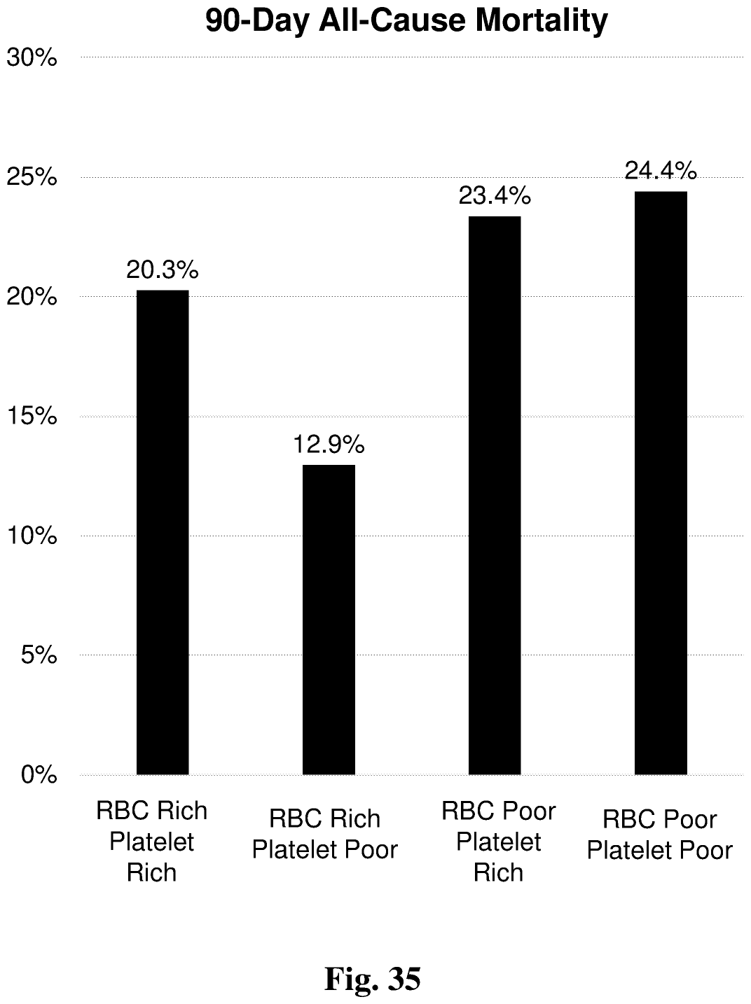

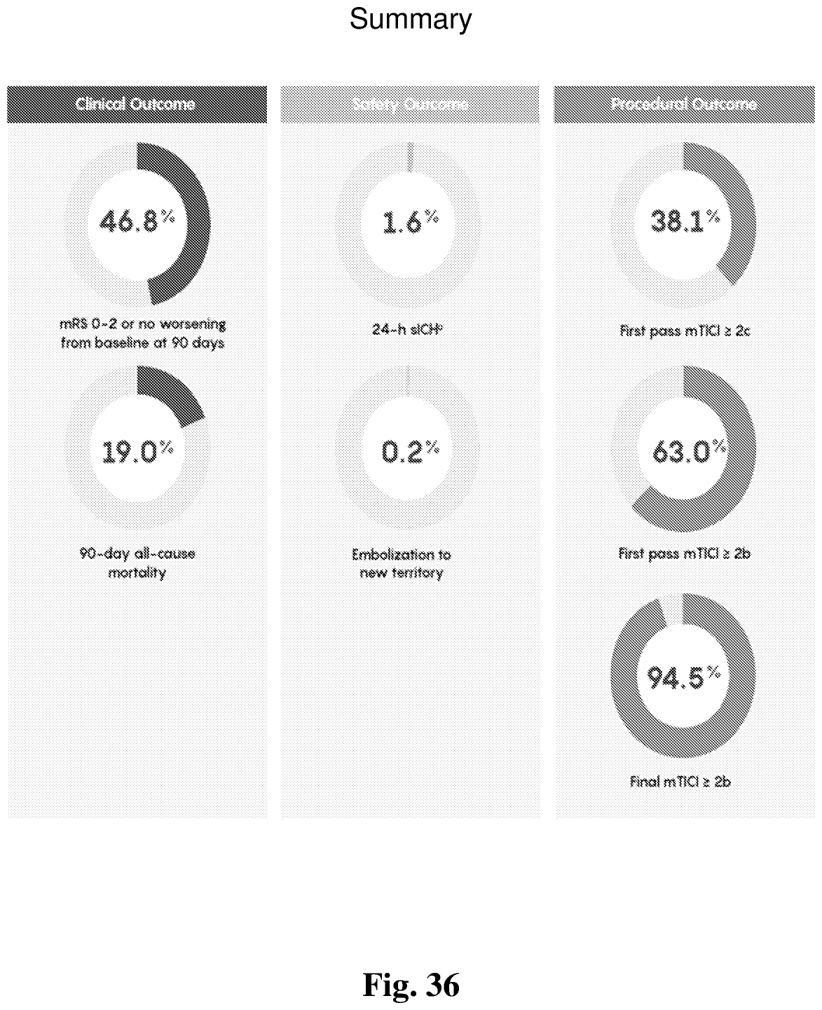

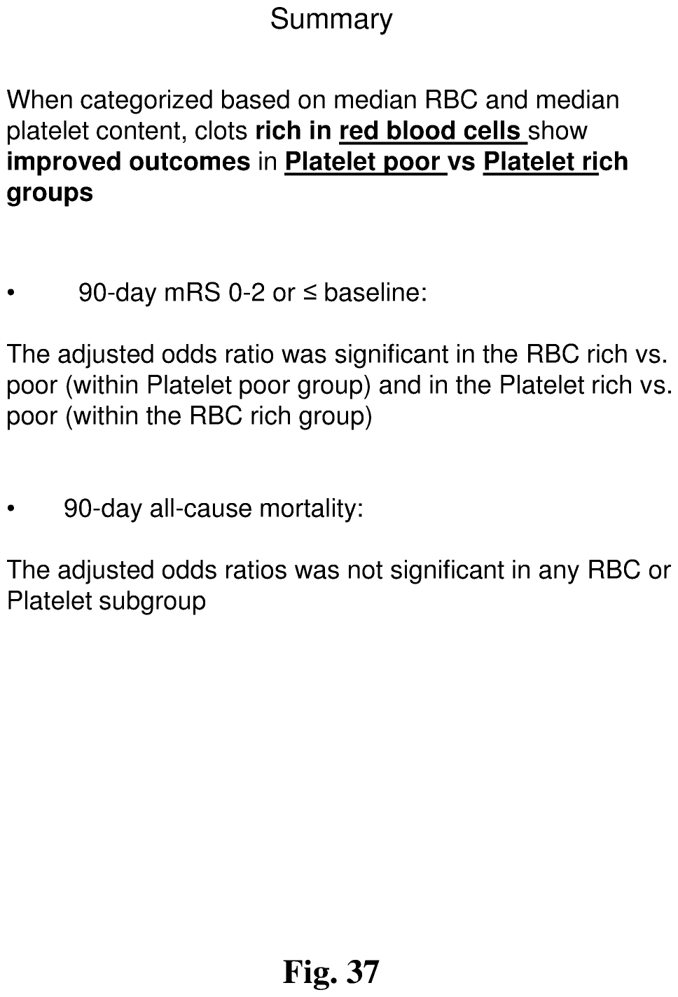

An analysis was also performed looking at clot composition as to whether it was RBC poor (<25%) or rich (>75%) with only the study device 200 regardless of passes. FIG. 29 outlines the results. The analysis of all clots over all patients and passes is outlined in FIG. 30. FIGS. 31-37 provide more analysis and content as to the role RBC plays in clot recovery and patient outcomes in mechanical thrombectomy.

The device 200 and related methods of use of this disclosure demonstrated high rates of substantial reperfusion and functional independence in patients with acute ischemic stroke secondary to large-vessel occlusions. The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

What is claimed is:

1. A method of restoring blood flow in neurovascular by removing thrombus in human patients experiencing ischemic stroke, the method comprising:
delivering a revascularization device to a blood vessel to retrieve a thrombus of a respective human patient of a plurality of human patients each having one or more cerebral occlusions; and
restoring perfusion to the blood vessel by passing the revascularization device by, through, or about the thrombus and removing the revascularization device to achieve, on a first pass of the revascularization device, an outcome of approximately a 63.2% revascularization rate with a cerebral infarction score of equal to or greater than a grade of 2b (mTICI ≥2b) for the plurality of human patients within a predetermined time period of natural stroke symptom onset.

2. The method of claim 1, further comprising: achieving approximately a 38.4% revascularization rate mTICI ≥2c on the first pass of the revascularization device in the blood vessel.

3. The method of claim 1, wherein the outcome is for a population size of the plurality of human patients that is at least 985 patients.

4. The method of claim 1, the revascularization device having a collapsed delivery configuration and an expanded deployed configuration, the revascularization device comprising:
a framework of struts forming a porous inner body flow channel and having a tubular main body portion and a distal end; and
a framework of struts forming an outer tubular body radially surrounding the tubular main body portion of the inner body during both the collapsed delivery configuration and the expanded deployed configuration, the inner body being connected to the outer tubular body.

5. The method of claim 4, the revascularization device further comprising:
a shaft extending between a proximal end and a distal end;
the outer body being self-expandable and coupled to the shaft, the self-expandable outer body comprising a plurality of longitudinally spaced clot scaffolding segments separated by voids forming one or more clot inlet mouths between adjacent clot scaffolding segments of the plurality of longitudinally spaced clot scaffolding segments; and
the inner body flow channel being self-expandable.

6. The method of claim 1, wherein the thrombus is located in one of the following locations: a carotid artery, a M1 middle cerebral artery, a M2 middle cerebral artery, a basilar artery, and a vertebral artery.

7. The method of claim 1, wherein the plurality of human patients experience a safety outcome of 1.6% based on a 24 hour symptomatic intracerebral hemorrhage (sICH) in cases with the first pass of the revascularization device.

8. The method of claim 7, wherein the safety outcome is for a population size of the plurality of human patients that is at least 997 patients.

9. The method of claim 1, wherein the plurality of human patients experience a safety outcome of 0.6% based on a 24 hour symptomatic intracerebral hemorrhage (sICH) in cases with a single pass of the revascularization device.

10. The method of claim 9, wherein the safety outcome is for a population size of the plurality of human patients that is at least 504 patients.

11. The method of claim 5, wherein the plurality of human patients experience a median number of passes of 1 in cases with the first pass of the revascularization device in a population size that is at least 997 patients and a final revascularization rate mTICI ≥2b in the blood vessel after procedure completion.

12. The method of claim 5, wherein 85.1% of the plurality of human patients in a population size that is at least 997 patients have less than or equal to 3 passes and a final revascularization rate mTICI ≥2b in the blood vessel after procedure completion.

13. The method of claim 1, wherein the outcome comprises a modified Rankin Scale (mRS) of 0 to less than or equal to 2 or less than or equal to baseline at 90 days of approximately 46.8%.

14. The method according to claim 5, inclusion criteria for the human patients comprising:
age ≥18y;
informed consent;
angiographic confirmation of LVO;
clinical decision for MT; and
the revascularization device planned as first attempted device/technique for MT.

15. The method according to claim 14, exclusion criteria for the human patients comprising:
currently participating in an investigational clinical trial that may confound study endpoints; and
pregnancy.

16. A method of restoring blood flow in neurovascular by removing thrombus in human patients experiencing ischemic stroke, the method comprising:
delivering a revascularization device to a blood vessel to retrieve a thrombus of a respective human patient of a plurality of human patients each having one or more cerebral occlusions; and
restoring perfusion to the blood vessel by passing the revascularization device by, through, or about the thrombus and removing the revascularization device to achieve, on a first pass of the revascularization device, a cerebral infarction score of between 2c and 3 (mTICI 2c-3) for the plurality of human patients, with the thrombus composition comprising a von Willebrand Factor percentage of 1.20 to 29.02.

17. The method of claim 16, wherein the thrombus composition comprises a von Willebrand Factor percentage of 15.11.

18. The method of claim 16, wherein the population size of the plurality of human patients is at least 192 patients.

19. The method of claim 16, wherein the population size of the plurality of human patients is at least 314 patients.
